SEQUENCE LISTING (32 sequences)

SEQ ID NO: 1 - DNA, 29 bp, Artificial
  Synthetic oligonucleotide
cagaattcaagcagcacgccaagggcaag

SEQ ID NO: 2 - DNA, 29 bp, Artificial
  Synthetic oligonucleotide
cagaattcgatgccgtcgtgctcctggtc

SEQ ID NO: 3 - DNA, 31 bp, Artificial
  Synthetic oligonucleotide
tattctagacatatgaccgttttggatgagg

SEQ ID NO: 4 - DNA, 24 bp, Artificial
  Synthetic oligonucleotide
acctctagacaacgctcgtggacc

SEQ ID NO: 5 - DNA, 27 bp, Artificial
  Synthetic oligonucleotide
ttatctagacatatgtcccctgccgac

SEQ ID NO: 6 - DNA, 32 bp, Artificial
  Synthetic oligonucleotide
atgaattctatgcatcgggtcagcgccagctg

SEQ ID NO: 7 - DNA, 30 bp, Artificial
  Synthetic oligonucleotide
atgaattctatgcatcgggtcagcgccagc

SEQ ID NO: 8 - DNA, 30 bp, Artificial
  Synthetic oligonucleotide
atgaattcatgcatgagggagcctcaatcg

SEQ ID NO: 9 - DNA, 26 bp, Artificial
  Synthetic oligonucleotide
agatctagatcagtccttgatctcgc

SEQ ID NO: 10 - DNA, 21 bp, Artificial
  Synthetic oligonucleotide
gctttgaggaccttggcgatg

SEQ ID NO: 11 - DNA, 21 bp, Artificial
  Synthetic oligonucleotide
gaagtacaggccgaagaccac

SEQ ID NO: 12 - DNA, 21 bp, Artificial
  Synthetic oligonucleotide
gcgatttcgccacgattggcg

SEQ ID NO: 13 - DNA, 21 bp, Artificial
  Synthetic oligonucleotide
ccgatatcagcccctgatgac

SEQ ID NO: 14 - DNA, 21 bp, Artificial
  Synthetic oligonucleotide
cgtcagcttgcccttggcgtg

SEQ ID NO: 15 - DNA, 21 bp, Artificial
  Synthetic oligonucleotide
ctacgctccgggtgagcgaac

SEQ ID NO: 16 - DNA, 31 bp, Artificial
  Synthetic oligonucleotide
ggagggccgtgatggcggcgacttcctcggg

SEQ ID NO: 17 - DNA, 35 bp, Artificial
  Synthetic oligonucleotide
gaggaactggtacgcgcgggcggtacaagcaagct

SEQ ID NO: 18 - DNA, 120 bp, Streptomyces coelicolor
aaacgggccggagactgtacggagtcgacggctcgcaatccttgctcggcttcgtagagtcgctacatgaccgttttggatgaggcgccgggcgagccgacggacgcgcgcgggcgggtg

SEQ ID NO: 19 - DNA, 120 bp, Streptomyces coelicolor
cacccgcccgcgcgcgtccgtcggctcgcccggcgcctcatccaaaacggtcatgtagcgactctacgaagccgagcaaggattgcgagccgtcgactccgtacagtctccggcccgttt

SEQ ID NO: 20 - protein, 18 aa, Streptomyces coelicolor
MTVLDEAPGEPTDARGRV

SEQ ID NO: 21 - DNA, 240 bp, Streptomyces coelicolor
cagccatgtctcacgagccggtaaccccggggcaaccgcgaaccggacggccccttccggcactggtcctgctccgcccggggggcgggtgattaccgcgggatggacgggcggctgaataccggccgatacgttctgcgcatgactgttccgaacagggcggcgtgaattccaaccgttggccgtcggcgagccccgatcagtaatcgagtgagtgaggagaatcttcgtgcgcaaggt

SEQ ID NO: 22 - DNA, 240 bp, Streptomyces coelicolor
ccacaaagaaaccgcgtggcccgcagcacgcccttacagagaccttgaccacacaggagggctagggtttcccccaggagtcctgcgtaccgcggtactacaagggctttcgggggtcgagcgagcctcgaatcacgctccgtgtgggcaagctcaccattggggacgggtcgaagtgccgtgtcggcagtccctaaactcggcttgtttcaaggagggagcctcaatcgtgcgcaaggt

SEQ ID NO: 23 - protein, 4 aa, Streptomyces coelicolor
VRVK

SEQ ID NO: 24 - DNA, 968 bp, Streptomyces coelicolor
tattctagacatatgaccgttttggatgaggcgccgggcgagccgacggacgcgcgcgggcgggtggccgagctgcacgggatccgtgcagcggcgctcgccgggccgagtgagaaggcgacggcggcgcagcacgccaagggcaagctgacggcacgtgagcgcatcgagctgctcctggaccccggctccttccgcgaggtcgagcagctgcgccggcaccgggcgaccgggttcggcctggaggccaagaagccgtacaccgacggtgtcatcaccggctggggcacggtcgagggccgcacggtcttcgtctacgcccacgacttccggatcttcggcggcgcgctgggcgaggcccacgccacgaagatccacaagatcatggacatggccatcgcggccggtgccccgctggtgtcgctgaacgacggcgccggcgcccgtatccaggagggcgtcagcgcgctcgccgggtacggcggcatcttccagcgcaacaccaaggcgtccggcgtcatcccgcagatcagcgtgatgctcggcccctgcgcgggcggcgcggcctacagccccgccctcaccgacttcgtcttcatggtccgcgacacctcgcagatgttcatcacgggcccggacgtcgtcaaggcggtcaccggcgaggagatcacgcagaacggtctgggcggcgccgacgtgcacgccgagacgtccggcgtgtgccacttcgcctacgacgacgaggagacctgcctcgccgaggtccgctacctcctctccctcctcccgcagaacaaccgggagaacccgccccgcgccgagtcctccgaccccgtggaccgccgctcggacaccctcctcgacctggtcccggcggacggcaaccgcccgtacgacatgaccaaggtcatcgaggaactcgtcgacgagggcgagtacctggaggtccacgagcgttgtctagaggt

SEQ ID NO: 25 - protein, 590 aa, Streptomyces coelicolor
VRKVLIANRGEIAVRVARACRDAGIASVAVYADPDRDALHVRAADEAFALGGDTPATSYLDIAKVLKAARESGADAIHPGYGFLSENAEFAQAVLDAGLIWIGPPPHAIRDRGEKVAARHIAQRAGAPLVAGTPDPVSGADEVVAFAKEHGLPIAIKAAFGGGGRGLKVARTLEEVPELYDSAVREAVAAFGRGECFVERYLDKPRHVETQCLADTHGNVVVVSTRDCSLQRRHQKLVEEAPAPFLSEAQTEQLYSSSKAILKEAGYGGAGTVEFLVGMDGTIFFLEVNTRLQVEHPVTEEVAGIDLVREMFRIADGEELGYDDPALRGHSFEFRINGEDPGRGFLPAPGTVTLFDAPTGPGVRLDAGVESGSVIGPAWDSLLAKLIVTGRTRAEALQRAARALDEFTVEGMATAIPFHRTVVRDPAFAPELTGSTDPFTVHTRWIETEFVNEIKPFTTPADTETDEESGRETVVVEVGGKRLEVSLPSSLGMSLARTGLAAGARPKRRAAKKSGPAASGDTLASPMQGTIVKIAVEEGQEVQEGDLIVVLEAMKMEQPLNAHRSGTIKGLTAEVGASLTSGAAICEIKD

SEQ ID NO: 26 - protein, 590 aa, Streptomyces coelicolor
VRKVLIANRGEIAVRVARACRDAGIASVAVYADPDRDALHVRAADEAFALGGDTPATSYLDIAKVLKAARESGADAIHPGYGFLSENADFAQAVLDAGLIWIGPPPHAIRDRGEKVAARHIAQRAGAPLVAGTPDPVSGADEVVAFAKEHGLPIAIKAAFGGGGRGLKVARTLEEVPELYDSAVREAVAAFGRGECFVERYLDKPRHVETQCLADTHGNVVVVSTRDCSLQRRHQKLVEEAPAPFLSEAQTEQLYSSSKAILKEAGYVGAGTVEFLVGMDGTISFLEVNTRLQVEHPVTEEVAGIDLVREMFRIADGEELGYDDPALRGHSFEFRINGDHPGRGFLPAPGTVTLFDAPTGPGVRLDAGVESGSVIGPAWDSLLAKLIVTGRTRAEALQRAARALDEFTVEGMATAIPFHRTVVRDPAFAPELTGSTDPFTVHTRWIETEFVNEIKPFTTPADTETDEESGRETVVVEVGGKRLEVSLPSSLGMSLARTGLAAGARPKRRAAKKSGPAASGDTLASPMQGTIVKIAVEEGQEVQEGDLIVVLEAMKMEQPLNAHRSGTIKGLTAEVGASLTSGAAICEIKD

SEQ ID NO: 27 - DNA, 1773 bp, Streptomyces coelicolor
gtgcgcaaggtgctcatcgccaatcgtggcgaaatcgctgtccgcgtggcccgggcctgccgggacgccgggatcgcgagcgtggccgtctacgcggatccggaccgggacgcgttgcacgtccgtgccgctgatgaggcgttcgccctgggtggtgacacccccgcgaccagctatctggacatcgccaaggtcctcaaagccgcgcgcgagtcgggcgcggacgccatccaccccggctacggattcctctcggagaacgccgagttcgcgcaggcggtcctggacgccggcctgatctggatcggcccgcccccgcacgccatccgcgaccgtggcgaaaaggtcgccgcccgccacatcgcccagcgggccggcgcccccctggtcgccggcacccccgaccccgtctccggcgcggacgaggtcgtcgccttcgccaaggagcacggcctgcccatcgccatcaaggccgccttcggcggcggcgggcgcggcctcaaggtcgcccgcaccctcgaagaggtgccggagctgtacgactccgccgtccgcgaggccgtggccgccttcggccgcggggagtgcttcgtcgagcgctacctcgacaagccccgccacgtggagacccagtgcctggccgacacccacggcaacgtggtcgtcgtctccacccgcgactgctccctccagcgccgccaccaaaagctcgtcgaggaggcccccgcgccctttctctccgaggcccagacggagcagctgtactcatcctccaaggccatcctgaaggaggccggctacggcggcgccggcaccgtggagttcctcgtcggcatggacggcacgatcttcttcctggaggtcaacacccgcctccaggtcgagcacccggtcaccgaggaagtcgccggcatcgacttggtccgcgagatgttccgcatcgccgacggcgaggaactcggttacgacgaccccgccctgcgcggccactccttcgagttccgcatcaacggcgaggaccccggccgcggcttcctgcccgcccccggcaccgtcaccctcttcgacgcgcccaccggccccggcgtccgcctggacgccggcgtcgagtccggctccgtcatcggccccgcctgggactccctcctcgccaaactgatcgtcaccggccgcacccgcgccgaggcactccagcgcgcggcccgcgccctggacgagttcaccgtcgagggcatggccaccgccatccccttccaccgcacggtcgtccgcgacccggccttcgcccccgaactcaccggctccacggaccccttcaccgtccacacccggtggatcgagacggagttcgtcaacgagatcaagcccttcaccacgcccgccgacaccgagacggacgaggagtcgggccgggagacggtcgtcgtcgaggtcggcggcaagcgcctggaagtctccctcccctccagcctgggcatgtccctggcccgcaccggcctggccgccggggcccgccccaagcgccgcgcggccaagaagtccggccccgccgcctcgggcgacaccctcgcctccccgatgcagggcacgatcgtcaagatcgccgtcgaggaaggccaggaagtccaggaaggcgacctcatcgtcgtactcgaggcgatgaagatggaacagcccctcaacgcccacaggtccggcaccatcaagggcctcaccgccgaggtcggcgcctccctcacctccggcgccgccatctgcgagatcaaggactga

SEQ ID NO: 28 - DNA, 1773 bp, Streptomyces coelicolor; identical to SEQ ID NO: 27

SEQ ID NO: 29 - DNA, 1584 bp, Streptomyces coelicolor
atgaccgttttggatgaggcgccgggcgagccgacggacgcgcgcgggcgggtggccgagctgcacgggatccgtgcagcggcgctcgccgggccgagtgagaaggcgacggcggcgcagcacgccaagggcaagctgacggcacgtgagcgcatcgagctgctcctggaccccggctccttccgcgaggtcgagcagctgcgccggcaccgggcgaccgggttcggcctggaggccaagaagccgtacaccgacggtgtcatcaccggctggggcacggtcgagggccgcacggtcttcgtctacgcccacgacttccggatcttcggcggcgcgctgggcgaggcccacgccacgaagatccacaagatcatggacatggccatcgcggccggtgccccgctggtgtcgctgaacgacggcgccggcgcccgtatccaggagggcgtcagcgcgctcgccgggtacggcggcatcttccagcgcaacaccaaggcgtccggcgtcatcccgcagatcagcgtgatgctcggcccctgcgcgggcggcgcggcctacagccccgccctcaccgacttcgtcttcatggtccgcgacacctcgcagatgttcatcacgggcccggacgtcgtcaaggcggtcaccggcgaggagatcacgcagaacggtctgggcggcgccgacgtgcacgccgagacgtccggcgtgtgccacttcgcctacgacgacgaggagacctgcctcgccgaggtccgctacctcctctccctcctcccgcagaacaaccgggagaacccgccccgcgccgagtcctccgaccccgtggaccgccgctcggacaccctcctcgacctggtcccggcggacggcaaccgcccgtacgacatgaccaaggtcatcgaggaactcgtcgacgagggcgagtacctggaggtccacgagcgttgggcccgcaacatcatctgcgcgctggcccgtctcgacgggcgggtcgtgggcatcgtcgccaaccagccgcaggccctggccggtgtcctggacatcgaggcgtcggagaaggcggcccgcttcgtccagatgtgcgacgccttcaacatcccgatcatcactcttctggacgtacccggcttcctgcccggcgtcgaccaggagcacggcgggatcatccgccacggcgccaagctgctctacgcgtactgcaacgcgaccgtgccccggatctcgctgatcctgcgcaaggcgtacggaggtgcttacatcgtcatggacagccagtccatcggcgccgacctcacctacgcctggccgaccaacgagatcgccgtcatgggcgcggaaggtgccgcgaacgtcatcttccgccggcagatcgccgacgccgaggaccccgaggccatgcgggcgcgcatggtcaaggagtacaagtccgagctgatgcacccctactacgcggccgaacgcggtctggtcgacgacgtcatcgaccccgccgaaacccgcgaggtgctgatcacgtccctggcgatgctccacaccaagcacgccgacctgccctcccgcaagcacggcaacccgccgcagtga

SEQ ID NO: 30 - DNA, 198 bp, Streptomyces coelicolor
atgtcccctgccgacatccgcgtcgagaagggccacgccgagcccgaggaagtcgccgccatcacggccctcctcctggcccgcgccgccgcccgccccgccgagatcgcgccgacccacggcggcggccgcgcccgcgccggctggcgccgcctggaacgcgagccgggcttccgcgccccgcacagctggcgctga

SEQ ID NO: 31 - protein, 527 aa, Streptomyces coelicolor
MTVLDEAPGEPTDARGRVAELHGIRAAALAGPSEKATAAQHAKGKLTARERIELLLDPGSFREVEQLRRHRATGFGLEAKKPYTDGVITGWGTVEGRTVFVYAHDFRIFGGALGEAHATKIHKIMDMAIAAGAPLVSLNDGAGARIQEGVSALAGYGGIFQRNTKASGVIPQISVMLGPCAGGAAYSPALTDFVFMVRDTSQMFITGPDVVKAVTGEEITQNGLGGADVHAETSGVCHFAYDDEETCLAEVRYLLSLLPQNNRENPPRAESSDPVDRRSDTLLDLVPADGNRPYDMTKVIEELVDEGEYLEVHERWARNIICALARLDGRVVGIVANQPQALAGVLDIEASEKAARFVQMCDAFNIPIITLLDVPGFLPGVDQEHGGIIRHGAKLLYAYCNATVPRISLILRKAYGGAYIVMDSQSIGADLTYAWPTNEIAVMGAEGAANVIFRRQIADAEDPEAMRARMVKEYKSELMHPYYAAERGLVDDVIDPAETREVLITSLAMLHTKHADLPSRKHGNPPQ

SEQ ID NO: 32 - protein, 65 aa, Streptomyces coelicolor
MSPADIRVEKGHAEPEEVAAITALLLARAAARPAEIAPTHGGGRARAGWRRLEREPGFRAPHSWR

(19) United States
(12) Patent Application Publication
(10) Pub. No.: US 2003/0157652 A1
(43) Pub. Date: Aug. 21, 2003

(54) Antibiotic production (II)

(76) Inventors: Hugo Cesar Gramajo, Rosario (AR); Eduardo Jose Rodriguez, Mountain View, CA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/045,612

(22) Filed: Oct. 23, 2001

Related U.S. Application Data
(60) Provisional application No. 60/242,533, filed on Oct. 23, 2000.

(51) Int. Cl. C07H 17/08; C07H 21/04; C12P 19/62; C12N 9/10; C12P 21/02; C12N 1/21; C12N 15/74

(52) U.S. Cl. 435/76; 536/23.2; 435/69.1; 435/252.3; 435/320.1; 435/193; 536/7.1

(57) ABSTRACT

The nucleic acid and amino acid sequences of α1, α2, β and ε subunits of acetyl-CoA carboxylase (ACCase) from Streptomyces coelicolor are provided. This subunit structure differs from that of known acyl carboxylases. Materials and methods are provided of increasing ACCase activity and production of secondary metabolites (such as polyketides and especially antibiotics) by causing expression in Streptomyces of such nucleic acid.

Also provided are methods of increasing ACCase activity and production of secondary metabolites (such as polyketides and especially antibiotics) by culturing Streptomyces in the presence of exogenous malonate.

### REFERENCE TO PROVISIONAL APPLICATION

This application claims the benefit of U.S. Provisional Application No. 60/242,533 filed on Oct. 23, 2000, the entire disclosure of which is incorporated by reference herein.

### INTRODUCTION

Malonyl-CoA is essential as a metabolic substrate in all living organisms studied so far and it also plays a role as a modulator of specific protein activity (for a review see Brownsey et al., 1997). Malonyl-CoA is a substrate for fatty acid synthase (FAS) (Bloch and Vance, 1977), for polyketide synthases (PKS) in plants, fungi and bacteria (Hopwood & Sherman, 1990) and for fatty acid chain-elongation systems (Saggerson, et al., 1992). Understanding the pathway(s) that lead to the biosynthesis of malonyl-CoA in Streptomyces might have an outstanding interest, since these micro-organisms are well known to have the ability to synthesize a vast array of pharmaceutically important polyketide compounds (such as antibiotic, antiparasitic, antifungal, immunosuppressant and/or antitumour polyketides), where malonyl-CoA is used as the most common extender unit (Hopwood & Sherman, 1990). Therefore, information gained on the enzyme(s) involved in the supply of this key metabolite will be relevant, not only for a better understanding of the primary metabolism of Streptomyces, but for improving production of many useful secondary metabolites.

Biosynthesis of malonyl-CoA occurs in most species through the ATP-dependent carboxylation of acetyl-CoA by an acetyl-CoA carboxylase (ACCase) (Bloch & Vance, 1977; Harwood, 1988). The overall reaction catalyzed by ACCase is a two step process that involves ATP-dependent formation of carboxybiotin followed by transfer of the carboxyl moiety to acetyl-CoA. The importance of this biosynthetic pathway is most directly reflected by the fact that ACCase expression is essential for normal growth of bacteria (Perez, et al., 1998; Li and Cronan, 1993), yeast (Hasselacher, et al., 1993) and isolated animal cells in culture (Pizer, et al., 1996).

Several complexes with ACCase activity have been purified from various actinomycetes. Interestingly, these complexes have also shown the ability to carboxylate other substrates like propionyl- and butyryl-CoA (Erfle, 1973; Henrikson and Allen, 1979; Huanaiti and Kolattukudy, 1982). This property has led to these enzyme being called acyl-CoA carboxylases, and all of them have been shown to consist of two subunits, a larger one (α-chain) with the ability to carboxylate its covalently bound biotin group, and a smaller sub-unit (β-chain) bearing the carboxyl transferase activity. However, there is no information gained, so far, regarding the physiological role of these enzymes.

In Streptomyces the purification of a complex with ACCase activity has proved to be unsuccessful, probably due to its high instability (Bramwell et al., 1996). However ACCase activity has been readily measured in crude extracts of S. coelicolor (Bramwell et al., 1996; Rodríguez and Gramajo, 1999), indicating that this enzyme complex was present in this micro-organism.

A pathway for the biosynthesis of malonyl-CoA in S. aureofaciens has been described that does not involve ACCase (Behal et al., 1977; Laakel et al., 1994). This route involves the anaplerotic enzymes phosphoenolpyruvate carboxylase and oxaloacetate dehydrogenase. In S. coelicolor A3(2), no evidence for the presence of oxaloacetate dehydrogenase has been found (Bramwell et al., 1993); thus, biosynthesis of malonyl-CoA in this organism seemed to occur exclusively through the ACCase enzyme activity.

Attempts carried on in S. coelicolor to characterize enzymes with carboxylase activity, have led to the characterization of two complexes exhibiting exclusively PCCase activity. The PCCase purified by Bramwell et al., (1996) comprises a biotinylated protein of 88 kDa, PccA, and a non-biotinylated component, the carboxyl transferase, of 66 kDa. More recently the inventors have also characterized at both the genetic and biochemical levels, the components of a second PCCase in this bacterium. In vitro reconstitution experiments have shown that an active complex could be obtained by mixing a carboxyl transferase component of 59 kDa (deduced MW, though it runs anomalously in SDS-PAGE, with an apparent MW of 65 kDa), PccB, with either of the two almost identical biotinylated components named AccA1 and AccA2 (Rodríguez and Gramajo, 1999). Recently a gene cluster encoding malonyl-CoA decarboxylase (MatA), malonyl-CoA synthetase (MatB) and a putative decarboxylate carrier protein (MatC) has been proposed as the pathway for malonate metabolism in Rhizobium trifolii (An and Kim, 1998). After the transport of the malonate by MatC, the malonate is converted into malonyl-CoA by MatB and finally decarboxylated to acetyl-CoA by MatA. However, the fact that the Kₘ of the malonyl-CoA decarboxylase for malonyl-CoA is relatively high has led the inventors to propose that malonyl-CoA synthesised from malonate by malonyl-CoA synthetase (rather than malonyl CoA synthesised by ACCase) is the major source for fatty acid biosynthesis in the bacterioid R. trifolii. Interestingly, genes with very high identity to MatC and MatB have been recently reported in the S. coelicolor genome project, suggesting that malonyl-CoA could also be synthesized from malonate in this micro-organism.

The inventors have identified an essential acyl-CoA carboxylase of S. coelicolor, and provide detailed genetic and biochemical characterization. The enzyme complex contains a unique sub-unit composition and appears to be the main pathway for the biosynthesis of malonyl-CoA, one of the key metabolites in the linkage between primary and secondary metabolism. An alternative pathway for the biosynthesis of malonyl-CoA is also proposed for cultures growing in malonate, and it most probably involves the matB and matC homologues of R. trifolii. However, even in these growing conditions, the acyl-CoA carboxylase seems to be essential for the viability of the micro-organism.

### SUMMARY OF INVENTION

Two genes accB and accE, forming a single operon, have been cloned from Streptomyces coelicolor A(3)2. The deduced amino acid sequence of AccB showed high similarity to carboxyl transferase of several propionyl- or acyl-CoA carboxylases of different actinomycetes. By contrast, AccE did not show any significant homology with protein sequences deposited in the GenBank data base. Heterologous expression of accB and accE in Escherichia coli and in vitro reconstitution of enzyme activity in the presence of the biotinylated component AccA1 or AccA2 confirmed that AccB was the carboxyl transferase subunit of an acyl-CoA carboxylase.

These experiments also established that AccE was a necessary component to obtain a fully active enzyme complex, whose subunit composition seems to be unique within this type of carboxylase. Gene disruption experiments clearly determined that AccB was essential for S. coelicolor viability. This protein together with AccA2, a biotinylated component essential for the viability of this micro-organism (Rodríguez and Gramajo, 1999), are the best candidates to form an acyl-CoA carboxylase, whose main physiological role is, most probably, the biosynthesis of malonyl-CoA.

Transcriptional studies of accBE, accA2 and accA1 have shown that accBE and accA2 are mainly expressed during vegetative and transition phase of growth, although some expression of these genes also occurred during stationary phase where they should provide the malonyl-CoA subunits for secondary metabolites biosynthesis. accA1 is only expressed during the transition phase of growth and its role in the formation of a carboxylase complex involved in providing the substrate for polyketide compounds of S. coelicolor is discussed.

Finally, an alternative route for the biosynthesis of malonyl-CoA is proposed when malonate is used as a carbon source. However, this route seems unable to substitute the main one, determined by the acyl-CoA carboxylase.

Accordingly, in a first aspect, the present invention provides a nucleic acid comprising a nucleic acid sequence which encodes an AccB polypeptide and/or an AccE polypeptide, or a nucleic acid sequence complementary thereto.

In a second aspect, the present invention provides a nucleic acid comprising a nucleic acid sequence which encodes an AccA1 and/or AccA2 polypeptide, or a nucleic acid sequence complementary thereto. It is believed that such nucleic acid was not made available to the public before Oct. 24, 1999, when the amino acid sequences of these polypeptides were disclosed in an oral presentation.

Exemplary nucleic acid sequences encoding the AccB, AccE, AccA1 and AccA2 polypeptides are given herein. Preferred embodiments of the invention include such sequences. However, it would be a matter of routine for the skilled person to obtain other nucleic acid sequences encoding these polypeptides, e.g. by introducing mutations which do not alter the encoded amino acid sequence, by virtue of the degeneracy of the genetic code, or by introducing mutations which alter the encoded amino acid sequence, within limits as defined below. Moreover, nucleic acids encoding variants of the polypeptides may be obtained e.g. by screening different strains of S. coelicolor or closely related species of Streptomyces using degenerate probes based on the sequences given herein.

Preferred nucleic acids of the first and second aspects encode AccB and AccE polypeptides along with an AccA1 and/or an AccA2 polypeptide (preferably AccA2).

The nucleic acid sequences encoding Acc polypeptides are preferably in operative association with regulatory sequences, e.g. sequences which enable constitutive or inducible expression in Streptomyces species. Examples of plasmids which include such regulatory sequences and of suitable promoters are given herein. A suitable inducible promoter is tipA (inducible by thiostrepton); suitable constitutive promoters are ermE and the optimised ermE*. Alternatively, naturally occurring nucleic acid sequences may be in operative association with the regulatory sequences with which they are normally associated, or corresponding regulatory sequences from homologous genes in other strains or species. For example, the nucleic acid sequences may be in operative association with the corresponding regulatory (e.g. promoter) sequences defined herein.

For detailed protocols relevant to this and other aspects, see standard reference texts, such as Sambrook et al. (1989) and Hopwood et al. (1985).

In a third aspect, the present invention separately provides AccB, AccE, AccA1 and AccA2 polypeptides having amino acid sequences encoded or encodable by the respective nucleic acid sequences referred to in the first and second aspects.

In a fourth aspect, the present invention provides: vectors containing the nucleic acids of the first and second aspects (preferably vectors, e.g. plasmids, suitable for transforming Streptomyces species for expression therein) and cells, particularly Streptomyces cells, transformed with such vectors. Furthermore, the present invention provides a method of producing a secondary metabolite of a Streptomyces species, the method comprising culturing such transformed Streptomyces cells and extracting the secondary metabolite from the cell culture. The metabolite may be purified and/or formulated as a commercial product according to standard procedures.

In a fifth aspect, the invention provides a method of modifying a secondary metabolite-producing strain of a Streptomyces species to increase production of said secondary metabolite, the method comprising modifying said strain to express, or to increase expression of, nucleic acid encoding one or more polypeptides selected from the group consisting of AccB, AccE, AccA1 and AccA2.

In a sixth aspect, the present invention provides a method of modifying a strain of a Streptomyces species to increase ACCase and/or PCCase activity, the method comprising modifying said strain to express, or to increase expression of, nucleic acid encoding one or more polypeptides selected from the group consisting of AccB, AccE, AccA1 and AccA2.

In a seventh aspect, the present invention provides a modified strain of a Streptomyces species, produced or producible according to the method of the fifth or sixth aspect. Also provided are cells of said strain, methods of producing secondary metabolites comprising culturing said cells and extracting the secondary metabolite, which may be purified and/or formulated as a commercial product.

In an eighth aspect, the invention provides a method of increasing production of a secondary metabolite in cells of a Streptomyces species, the method comprising culturing said cells in the presence of exogenous malonate, preferably at a concentration of at least about 0.1%, more preferably at least about 0.2%, 0.4%, 0.5%, 0.75% or 1%, though higher concentrations may be used. 1% represents 1 g per 100 ml of medium.

### DETAILED DESCRIPTION

In relation to the fifth and sixth aspects, the modification preferably provides for increased expression of nucleic acid encoding more than one of AccE, AccE, AccA1 and AccA2, more preferably at least AccB and AccE or at least AccB and either AccA1 or AccA2, more preferably AccB, AccE and either AccA1 or AccA2. Of AccA1 and AccA2, AccA2 is preferred. Increased expression of nucleic acid encoding both AccA1 and AccA2 (usually in combination with AccB and optionally AccE) is also contemplated.

The methods of the fifth and sixth aspects preferably include a step of transforming a Streptomyces cell with a said nucleic acid under the control of a constitutive or inducible promoter, preferably a strong promoter. However, the expression of existing said nucleic acid could be increased, e.g. by placing them under the control of a stronger promoter sequence or sequences.

Exogenous said nucleic acid can replace existing said nucleic acid in the cell, or can be added without removing or functionally deleting existing said nucleic acid.

Acc Polypeptides and Acc Genes

In the definitions herein of the invention, and of the scope of protection (but not, except where the context requires otherwise, in the experimental sections), the term AccB is intended to include not only a polypeptide having the deduced amino acid sequence encoded by the nucleic acid sequence of FIG. 12 (though this is a preferred embodiment), but also a polypeptide which is a variant (e.g. an allelic or isoallelic variant) or a derivative of said polypeptide, having at least about 60% amino acid identity with said polypeptide, preferably at least about 65%, 70% or 75%, especially preferably (in view of the similarity of AccB as disclosed herein to another protein of unconfirmed function) at least about 80%, 85%, 90%, 92%, 94%, 96%, 98% or 99% identity. Such a variant or derivative may possess any one or more of the biological properties of the wild-type AccB protein, as disclosed herein, e.g. complex formation with AccA1, AccA2 and/or AccE (or allosteric regulation by AccE), ACCase and/or PCCase activity when AccB is co-expressed with AccA1, AccA2 and/or AccE, or increased secondary metabolite production when AccB is overexpressed in Streptomyces species (preferably in conjuction with AccA1, AccA2 and/or AccE).

Similarly, the term AccE is intended to include not only a polypeptide having the deduced amino acid sequence encoded by the nucleic acid sequence of FIG. 13 (though this is a preferred embodiment), but also a polypeptide which is a variant (e.g. an allelic or isoallelic variant) or a derivative of said polypeptide, having at least about 40% amino acid identity with said polypeptide, preferably at least about 50%, 60%, 70%, 80%, 85%, 90%, 95% or 99% identity. Such a variant or derivative may possess any one or more of the biological properties of the wild-type AccE protein, as demonstrated herein, e.g. complex formation with AccA1, AccA2 and/or AccB (or allosteric regulation of AccB), ACCase and/or PCCase activity when AccE is co-expressed with AccB, or increased secondary metabolite production when AccE is overexpressed in Streptomyces species (preferably in conjuction with AccB).

Similarly, the terms AccA1 and AccA2 are intended to include not only the polypeptides having the amino acid sequences shown in FIG. 11 (though these are respective preferred embodiments), but also polypeptides which are variants (e.g. allelic or isoallelic variants) or are derivatives of said polypeptides, having at least about 75% amino acid identity with said polypeptide, preferably at least about 80%, 85%, 90%, 92%, 94%, 96%, 98% or 99% identity. Such variants or derivatives may possess any one or more of the biological properties of the wild-type AccA1 or AccA2 polypeptides, as disclosed herein, e.g. complex formation with AccB and/or AccE, ACCase and/or PCCase activity when AccA1 or AccA2 is co-expressed with AccB and/or AccE, or increased secondary metabolite production when AccB is overexpressed in Streptomyces species (preferably in conduction with AccB and/or AccE).

A variant or a derivative of a given peptide may have one or more of internal deletions, internal insertions, terminal truncations, terminal additions, or substitutions of one or more amino acids, compared to the given peptide.

References to nucleic acid encoding AccA1, AccA2, AccB and/or AccE (or to accA1, accA2, accB and/or accE genes) should be interpreted accordingly.

In relation to the first aspect, preferred nucleic acids comprise a nucleic acid sequence having at least about 50%, preferably at least about 60%, 70%, 80%, 85%, 90%, 95%, 98% or 99% nucleic acid sequence identity with the accB nucleic acid sequence shown in FIG. 12. Other preferred nucleic acids comprise a nucleic acid sequence having at least about 40%, preferably at least about 50%, 60%, 70%, 80%, 85%, 90%, 95%, 98% or 99% nucleic acid sequence identity with the accE nucleic acid sequence shown in FIG. 13. Similarly, in relation to the second aspect, preferred nucleic acids comprise a nucleic acid sequence having at least about 50%, preferably at least about 60%, 70%, 80%, 85%, 90%, 95%, 98% or 99% nucleic acid sequence identity with the accA1 or accA2 nucleic acid sequence shown in FIG. 11.

Secondary Metabolites and Streptomyces Species

While the experimental disclosure herein relates to the production of Act (actinomycin) and Red (undecylprodigiosin) in S. coelicolor A3(2) (strain M145), it is thought that the teaching is applicable to other strains of Streptomyces in particular, it is thought that overexpession of all three Acc polypeptides (i.e. AccB, AccE and AccA1 and/or AccA2) will lead to increased malonyl-CoA production in substantially any Streptomyces species or even in other actinomycetes or in fungi (which also produce polyketide compounds). Since malonyl-CoA is an essential metabolic substrate, it is thought that this will lead to greater yield of desired secondary metabolites (for which see page 1), e.g. polyketides (including antibiotic polyketides) and fatty acids.

Preferred secondary metabolites are, however, antibiotics, especially Act and Red.

Preferred Streptomyces species are the closely related species S. coelicolor, S. violaceoruber, S. lividans and S. parvulus, especially S. coelicolor. Strains of such species are commonly available, e.g. from the ATCC, for example under ATCC deposit numbers 12434 for S. parvulus and 19832 for S. violaceoruber. S. coelicolor A3(2) and S. lividans 66 are available from the John Innes Culture Collection (Norwich, UK) under JICC deposit numbers 1147 and 1326, respectively. However, the invention is not limited to such particular strains.

Acetyl-CoA

In preferred embodiments, present invention further provides for the increased production in Streptomyces of acetyl-CoA, since it is thought that when ACCase activity is increased by the methods and means of the present invention, production of malonyl-CoA may become limited by the availability of the substrate acetyl-CoA. It is proposed that increased acetyl-CoA production could then lead to a further increased rate of malonyl-CoA production and hence secondary metabolite production. For example, oils or fatty acids could be used as the carbon source (together with glucose); fatty acids are degraded by b-oxidation giving high levels of acetyl-CoA.

Sequence Identity

“Percent (%) amino acid sequence identity” is defined as the percentage of amino acid residues in a candidate sequence that are identical with the amino acid residues in the sequence with which it is being compared, after aligning the sequences and introducing gaps, if necessary, to achieve the maximum percent sequence identity, and not considering any conservative substitutions as part of the sequence identity. The % identity values used herein are generated by WU-BLAST-2 which was obtained from Altschul et al. (1996); http://blast.wustl/edu/blast/README.html. WU-BLAST-2 uses several search parameters, most of which are set to the default values. The adjustable parameters are set with the following values: overlap span=1, overlap fraction=0.125, word threshold (T)=11. The HSPS and HSPS2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region, multiplied by 100. The “longer” sequence is the one having the most actual residues in the aligned region (gaps introduced by WU-BLAST-2 to maximize the alignment score are ignored).

“Percent (%) nucleic acid sequence identity” is defined as the percentage of nucleotide residues in a candidate sequence that are identical with the nucleotide residues in the sequence under comparison. The identity values used herein were generated by the BLASTN module of WU BLAST-2 set to the default parameters, with overlap span and overlap fraction set to 1 and 0.125, respectively.

Culture and Purification

Methods of genetic manipulation, cell culture and purification of expression products produced in cell culture are well known to the skilled person, e.g. from standard textbooks such as Sambrook et al (1989). In particular, methods for genetically manipulating Streptomyces, culturing Streptomyces under conditions suitable for secondary metabolite (e.g. polyketide and/or antibiotic production) and purifying secondary metabolites from Streptomycete cell culture medium are well known, e.g. from Hopwood et al. (1985) and Kieser et al (2000).

Formulation

Similarly, methods of formulating active compounds (e.g. polyketides, particularly antibiotics) as pharmaceuticals are well known in the art. Such pharmaceutical formulations may comprise, in addition to the active compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, transdermal, transmucosal, intramuscular, intraperitoneal routes.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active compound will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Formulations suitable for transmucosal administration include liquids, solutions, suspensions, emulsions, suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Ointments are typically prepared from the active compound and a paraffinic or a water-miscible ointment base.

Creams are typically prepared from the active compound and an oil-in-water cream base. The aqueous phase of the cream base may include at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Formulations may suitably be provided as a patch, adhesive plaster, bandage, dressing, or the like which is impregnated with one or more active compounds and optionally one or more other pharmaceutically acceptable ingredients, including, for example, penetration, permeation, and absorption enhancers.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences (supra).

A pharmaceutical formulation may be administered alone or in combination with other treatments, either simultaneously or sequentially dependent upon the condition to be treated.

The work underlying the invention will now be described in detail, by way of example only, with reference to the accompanying figures.

### FIGURES

FIG. 1 Organization of the genomic region of S. coelicolor M145 chromosome harbouring accB and accE genes. A. Genetic and physical map of the 6.2 kb insert in pRM08. The secondary structure downstream accE represents a rho-independent transcriptional terminator. Fragments I and II were amplified by PCR with the pair of oligos accBup-accBdown and accBEup-accBEdown respectively, uniquely labelled at the 5′-end (*) and used as probes in transcriptional analysis of the accBE operon. B. Physical map of the DNA fragments cloned in pET22b(+) and used for the heterologous expression of accB and/or accE. Only the most relevant restriction sites are shown: B, BamHI; Bc, BclI; E, EcoRI; K, KpnI; Nd, NdeI; N, NotI; S, SpHI.

FIG. 2 Attempted disruption of accB. A. Diagram showing the integration of pTR124 through one of the accBE flanking regions and the resolution of the cointegrate by a second event of homologous recombination. The crossed out arrow indicates the impossibility of obtaining the replacement of the wild-type accB by the Hyg^{R} mutant allele. B. The integration of a second copy of the accBE genes in the ΦC31 att site of T124 (to yield strain T149) allowed the replacement of the wild-type accB by a mutant allele containing the Hyg resistance cassette.

FIG. 3 Growth-phase dependent expression and transcription start site of the accBE operon. A. S1 nuclease mapping of accB, actII-ORF4 and hrdB, using RNA isolated from a liquid time course of S. coelicolor M145. Exp, Trans and Stat indicate the exponential, transition and stationary phase of growth, respectively. B. The nucleotide sequences of both strands from the accB promoter region are shown. The arrow indicates the most likely transcription start point for the accBE promoter, as determined by S1 nuclease mapping. The potential -10 and -35 regions for the accBEp are underlined. C. S1 nuclease mapping of the accB accE intergenic region using a 563 nt probe. FLP represents the full-length RNA-protected fragment that is 13 nt shorter than the probe.

FIG. 4 Growth-phase dependent expression of accA2 and accA1. S1 nuclease mapping of accA2 (A) and accA1 (B), using RNA isolated from a liquid time course of S. coelicolor M145.

FIG. 5 Mapping of the accA2 and accA1 transcription start point. A. High resolution S1 nuclease mapping of the 5′end of the accA2 transcript. S1, RNA-protected products of the S1 nuclease protection assay. Lanes labelled A, C, G and T indicate a dideoxy sequencing ladder using the same oligonucleotide that was used to make the S1 probe (accA2down). B. High resolution S1 nuclease mapping of the 5′ end of the accA1 transcript. S1, RNA-protected products of the S1 nuclease protection assay. Lanes labelled T, G, C and A indicate a dideoxy sequencing ladder using the same oligonucleotide that was used to make the S1 probe (accA1down). C. Sequence of the accA2 and accA1 upstream regions, indicating the most likely transcription start points for the promoters of each of the accA1 and accA2 genes (bent arrows). The potential -10 and -35 sequences for the accA1 and accA2 promoters are underlined. The potential ribosomal binding sites (rbs) are highlighted with bold letters. The 16 nt direct repeats (DR) found upstream of the transcription start point of accA1p1 are indicated with straight arrows.

FIG. 6 Construction and analysis of the accBE conditional mutant. A. Diagram showing the integration of pIJ8600 in strain M86 and the expected organisation of the Campbell integration of pTR94 in M94. Restriction sites: B. BamHI; N, NotI; Nd, NdeI;

S, SacI; Sp, SphI; Xb, XbaI. B. Hybridisation analysis of Southern blot of SacI-digested DNAs from M145, M86 and M94. The probe was the internal NdeI-XbaI fragment of accB shown in A (see FIG. 10).

FIG. 7 Expression of the acyl-CoA components in M86 and M94. A. SDS-PAGE of cell-free extracts of S. coelicolor M86 and M94 strains grown in YEME medium containing 10 μg/ml Am with or without the addition of 5 μg/ml Th. B. A duplicate of the SDS-PAGE gel shown in A was subjected to Western blotting and stained for biotinylated proteins by using alkaline phosphatase-streptavidin conjugate.

FIG. 8A Growth curves of M145, M86 and M94 strains. 10⁸ spores of strains M86 and M94 were inoculated in YEME medium containing 10 μg of Am or 10 μg/ml Am and 5 μg/ml of Th. 10⁸ spores of M145 were inoculated in YEME. The growth was followed by measuring OD_{450 nm}.

FIG. 8B Actinorhodin production in M94 and M145 in cultures grown in the presence of 5 μg of Th.

FIG. 9 Morphological and physiological differentiation of M86 and M94 in the presence of Th. Spores of M86 and M94 were spread in R2 or R5 medium containing 10 μg/ml Am. A drop containing 1 μg of Th was spotted in the centre of each plate. The picture shows the results obtained after the incubation of the plate at 30° C. for 48 h.

FIG. 10 The sequence of the amplification product obtained from accB using primers TC16 and TC17. NdeI (CATATG) and XbaI (TCTAGA) sites introduced into the accB by the primers are shown in bold. The 1 kb NdeI-XbaI fragment was cloned into pIJ8600.

FIG. 11 A. Amino acid sequences and B. Nucleic acid sequences of accA1 and accA2.

FIG. 12 A. Amino acid sequence and B. Nucleic acid sequence of accB.

FIG. 13 A. Amino acid sequence and B. Nucleic acid sequence of accE.

FIG. 14 Plasmid map for the construction of an expression vector for accA, accB and accE.

### EXAMPLE 1

### Cloning of AccBE Genes

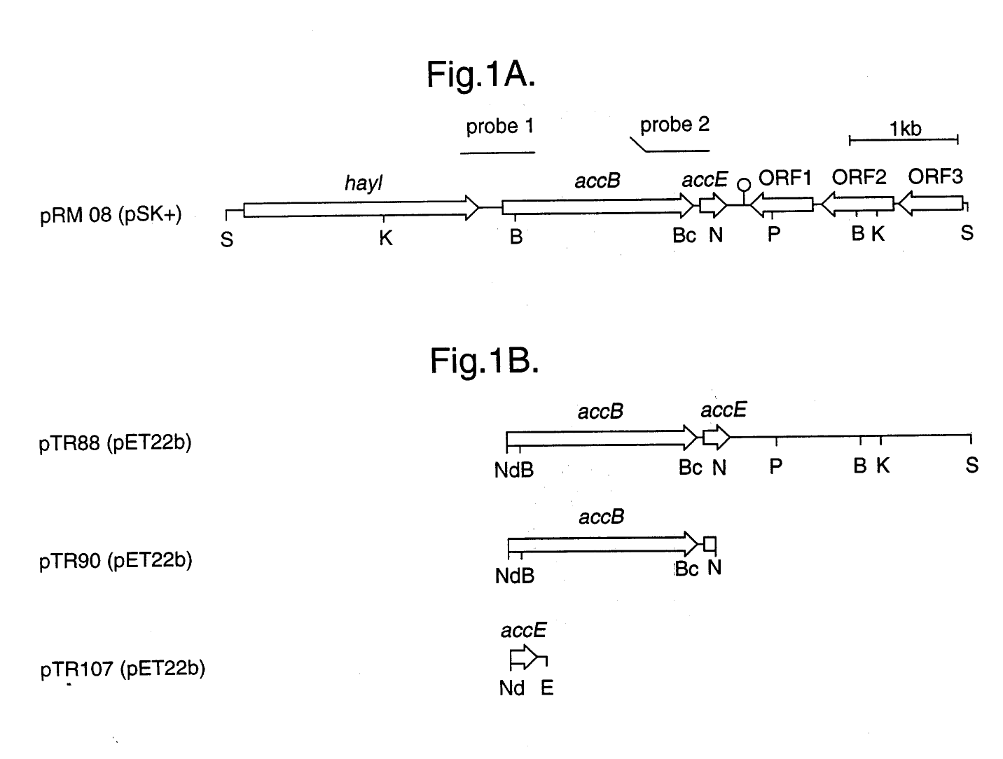

pccB of S. coelicolor (Rodríguez and Gramajo, 1999) was used as an heterologous probe in Southern blot experiments. When a BamHI digest of S. coelicolor DNA was probed with pccB and washed under low stringent conditions, a second, low hybridising, band was readily detected (data not shown). The target sequence was cloned from a size-enriched library as a 2.5 kb BamHI fragment and sequenced as described in Experimental Procedures (below). The sequence revealed the presence of an incomplete ORF with high homology to pccB. The complete gene was finally cloned as a 6 kb SstI fragment yielding pRM08 (FIG. 1). Sequencing and analysis of this DNA fragment revealed the presence of an ORF that exhibited end-to-end similarity with a putative decarboxylase (though the real function is unknown) of S. cyanogenous (Westrich et al., 1999), with the S. coelicolor PccB (Rodríguez and Gramajo, 1999) and with the β-subunit (PccB) of the Sac. erythraea PCCase (Donadio, et al., 1996). The levels of identity were 76%, 57% and 56%, respectively. The gene encoding this new putative carboxyl transferase was called accB.

Surprisingly, the sequence also revealed the presence of a small ORF, designated accE, whose start codon is only 17 bp downstream of the termination codon of accB. A 17 nt inverted repeat, which could function as a factor-independent bidirectional transcriptional terminator (reviewed in Lewin, 1994), separates accE from three convergent ORFs with homology to putative proteins of M. tuberculosis with unknown functions. The putative AccE polypeptide has a deduced molecular mass of 7.07 kDa and no significant homology to this polypeptide was found in a search of sequences deposited in the GenBank database.

Upstream of accB there is an ORF highly homologous to several known hialuronidases.

### EXAMPLE 2

### AccB is Essential for S. coelicolor Viability

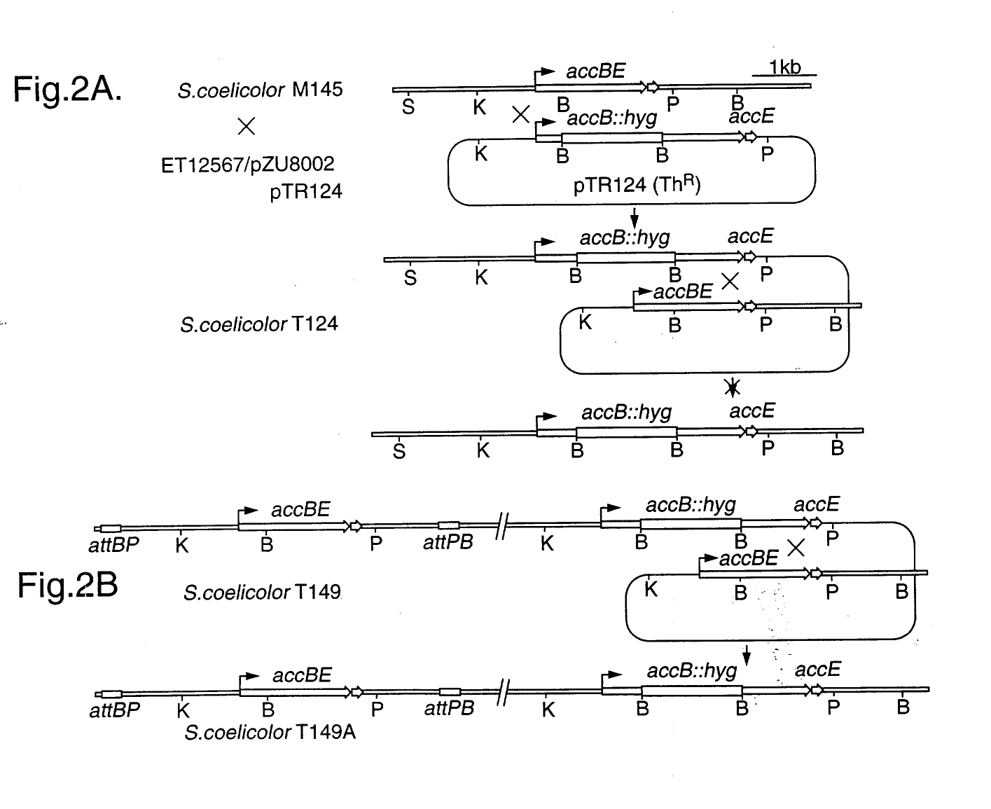

An accB mutant was constructed by gene replacement (FIG. 2A). A Hyg-resistant cassette was cloned in the unique BamHI site present in the coding sequence of accB, contained in PTR80. After an intermediate construction in pIJ2925, a BglII fragment containing the mutated allele was finally cloned in the conjugative vector pSET151. The resulting plasmid, pTR124, was cloned into the E. coli donor strain ET12567/pUZ8002 and transferred by conjugation into M145. Exconjugants were selected for Th^{R} Hyg^{R} for a simple crossover event. One of the exconjugants, named T124, was taken through four rounds of non-selective growth (SFM Hyg) to promote homologous recombination for the second crossover. Spores were plated to give single colonies and several thousands screened for Th sensitivity (which would have reflected successful gene replacement), but no Th^{S} isolates were obtained. This result suggested that accB is essential for S. coelicolor viability.

The inventors proposed, however, that if a second copy of accB were present in the chromosome of T124, a second crossover event (leading to the replacement of the wild type gene by the Hyg^{R} mutant allele) would then be allowed. To confirm this hypothesis, pTR149, which contains a copy of the accBE genes under its own promoter (see Experimental procedures, FIG. 2B), was first integrated in the ΦC31 attB site of T124. (The introduction of a second copy of both genes into the chromosome was prompted by the probability of a polar effect on accE taking place after the gene replacement event and because AccE is important for the recovery of a fully active acyl-CoA carboxylase complex—see in vitro reconstitution experiments below). The resultant strain T149 (Hyg^{R}, Th^{R}, Am^{R}) was passed through three rounds of sporulation on SFM Hyg Am and after the screening of approximately 500 colonies, 20 were found to be Am^{R} Hyg^{R} Th^{S}. The final chromosomal organization of accB in each of the strains constructed (T124, T149 and T149A), was analyzed by Southern blots using an internal fragment of accB as a probe.

### EXAMPLE 3

### Heterologous Expression of AccB, AccE and In Vitro Reconstitution of an Acyl-CoA Carboxylase Complex

Since accB proved to be essential for S. coelicolor viability, we could not clearly evaluate in vivo the physiological function of this gene product.

In order to study if AccB and AccE were components of an acyl-CoA carboxylase complex, we attempted in vitro reconstitution of the enzyme activity by mixing E. coli cell-free extracts containing the AccB and AccE with cell-free extracts containing the biotinylated sub-units AccA1 or AccA2. E. coli does not contain an ACCase enzyme, so ACCase activity cannot be assayed directly by carboxylation of acetyl-CoA (Polakis et al., 1972); therefore, the acyl-CoA carboxylase activity measured in these crude extracts exclusively represents the activity of the heterologous complexes reconstituted in vitro.

Heterologous expression of accB and accE was attempted by introducing a NdeI site at the ATG start codon of accB; after an intermediate construction (see Experimental procedures), accBE was cloned as a NdeI-SacI fragment into pET22(b), yielding pTR88 (FIG. 1). Transformation of E. coli BL21(DE3) with this plasmid yielded strain RG8 (Table 1). Crude extracts of RG8, prepared from IPTG-induced cultures, showed a clear over-expression of a 64 kDa protein in a 15% SDS-PAGE, corresponding to AccB; by contrast, AccE was not clearly visualised by Coomassie blue staining of the same gel (data not shown). In vitro reconstitution of an acyl-CoA carboxylase was then attempted my mixing crude extracts prepared from IPTG-induced cultures of RG8 with cell-free extracts of the E. coli strains RG7, which overproduces the biotinylated protein AccA1. After incubation for 1 h at 4° C., the mixture was assayed for ACCase and PCCase activity. As shown in Table 2 an enzyme complex showing high levels of both ACCase and PCCase activities was successfully reconstituted

To study if cell-free extracts containing AccB but not AccE were capable of reconstituting an active acyl-CoA carboxylase complex when mixed with cell-free extracts containing AccA1, we constructed a new pET22(b) derivative that only expresses accB. For this we took advantage of the NotI site present approximately in the middle of the coding sequence of accE and cloned the NdeI-NotI fragment from pTR88 into the expression vector, yielding pTR90 (FIG. 1).

Cell-free extracts of RG9, obtained by transformation of BL21(DE3) with pTR90, showed high levels of soluble AccB after IPTG induction. However, the acyl-CoA carboxylase complex reconstituted in vitro, after mixing cell-free extracts of RG9 (AccB) and RG7 (AccA1), showed much lower levels (approximately 10%) of ACCase and PCCase activities than the acyl-CoA carboxylase previously obtained by mixing RG8 with RG7 cell-free extracts (Table 2). Since the levels of AccB in cell-free extracts of RG8 and RG9 were essentially the same, we inferred from these experiments that AccE was necessary in order to obtain a fully active acyl-CoA carboxylase complex.

To confirm that the absence of AccE was the responsible of the lower acyl-CoA carboxylase activities, we studied the effect that the addition of cell-free extract containing AccE, had on the crude extracts containing AccB and AccA1 proteins. For this we first constructed strain RG10 (BL21(DE3) containing pTR107) that expresses high levels of soluble AccE (data not shown).

When cell-free extracts of RG10 where mixed with those of RG9 (AccB) and RG7 (AccA1) and incubated for 1 h on ice, the levels of enzyme activity where at least five times higher than in the control experiment, without the addition of AccE (Table 2). Although the results presented in this section clearly show that AccE is a functional part of the acyl-CoA carboxylase, enzyme kinetics studies with purified components will be necessary to understand more precisely the role of this protein in the enzyme complex activity. Similar results were obtained in all the reconstitution experiments mentioned above when AccA1 was replaced by AccA2 as the biotinylated component of the acyl-CoA carboxylase, indicating that either AccA1 or AccA2 can be efficiently used as the α-subunit of this enzyme complex.

### EXAMPLE 4

### Transcriptional Analysis of AccBE, AccA1 and AccA2

At least four combinations that resulted in active carboxylase complexes have been reconstituted by mixing the β-subunits PccB (Rodríguez and Gramajo, 1999) or AccB (this work) with either of the two almost identical α-subunits, AccA1 or AccA2. In any of these complexes the carboxyl transferase subunit seems to dictate the substrate specificity; thus, PccB seems to recognize only propionyl-CoA, while AccB has a broader substrate specificity, which allows the enzyme to recognize either acetyl- or propionyl-CoA. Moreover, a third complex with PCCase activity has also being described in S. coelicolor (Bramwell, et al., 1996). These findings show a remarkable overlapping of gene function in Streptomyces species. We followed two different approaches to gain more information on this; one was the generation of mutants and the second the study of the mRNA levels of some of these four genes throughout the different growth stages by using S1 nuclease protection.

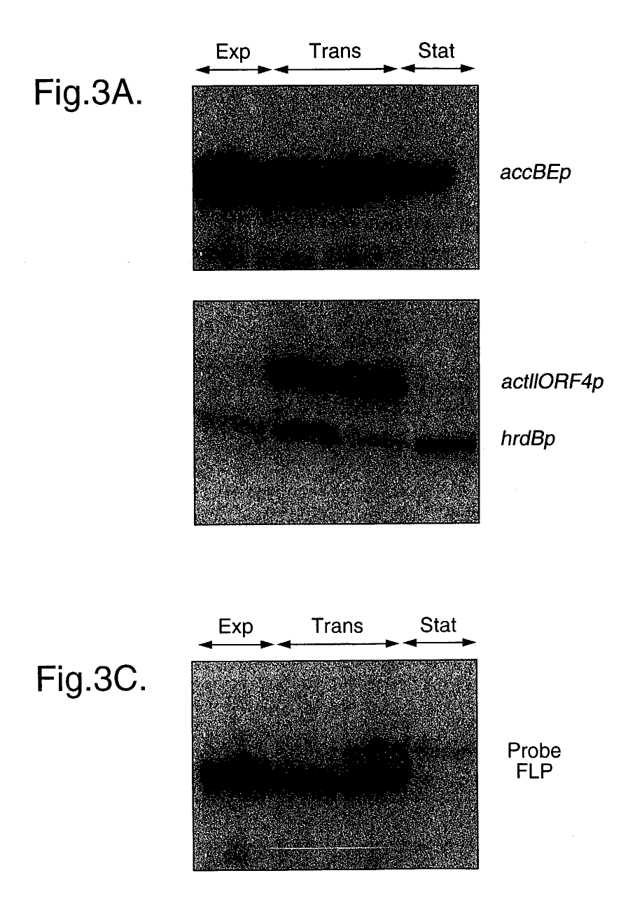

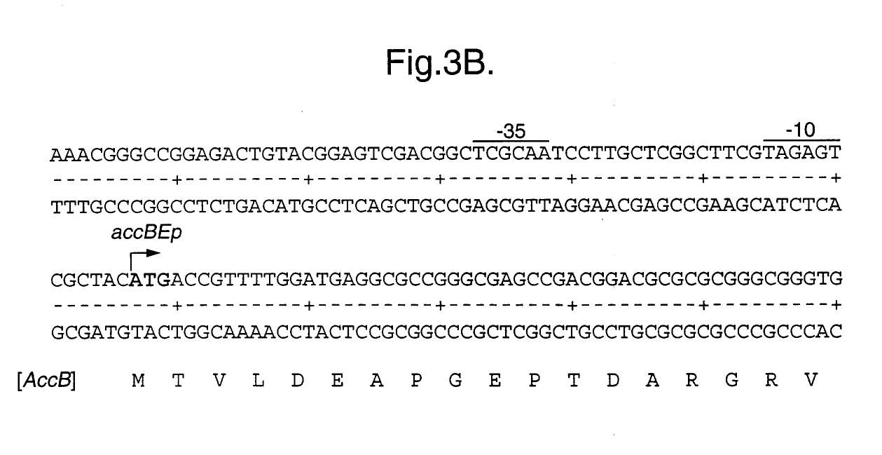

S. coelicolor A3(2) strain M145 was grown in SMM medium and RNA extracted at exponential, transition and stationary phase. S1 nuclease protection of accB was performed by using a 483 bp PCR product, uniquely labelled at the 5′end of the downstream oligo. Transcription of accB occurs primarily during active growth (exponential and transition phases), while its level of expression decayed significantly after entering into stationary phase (FIG. 3A). The transcripts of the major essential sigma factor hrdB and of the pathway-specific activator gene for acitnorhodin biosynthesis, actII-ORF4, were also studied as positive controls for the RNAs used in these experiments. As expected from previous results, hrdB was expressed constantly throughout growth (Buttner, M. J., 1990), while actII-ORF4 had a peak of expression during transition phase that shut off in stationary phase (Gramajo, et al., 1993).

The RNA-protected fragments found for accB corresponded to a transcription start site 1 bp upstream, or in the adenine, of the most likely translation start site of accB. Upstream of the transcription initiation site we found a putative -10 and -35 promoter regions with a high consensus sequences of promoters recognised by the vegetative σ^{hrdB} (Strohl, 1991) (FIG. 3B).

In order to find out if accB and accE were co-transcribed as a unique bi-cistronic mRNA, a new 563 bp probe was obtained by PCR. For this we used a 5′oligo corresponding to a sequence within the coding region of accB and a 3′oligo corresponding to a sequence within accE. The full-length RNA-protected fragment was easily differentiated from the probe-probe re-annealing due to the addition of a 13 nt tail to the 5′oligonucleotide (Experimental Procedures). The results obtained in this experiment clearly showed that accB and accE were part of the same transcript, confirming that these two genes form a single-copy operon (FIG. 3C). Moreover, the expression of accBE during the different growth phases as detected with this new probe followed the same profile as the expression observed with the probe used for accB.

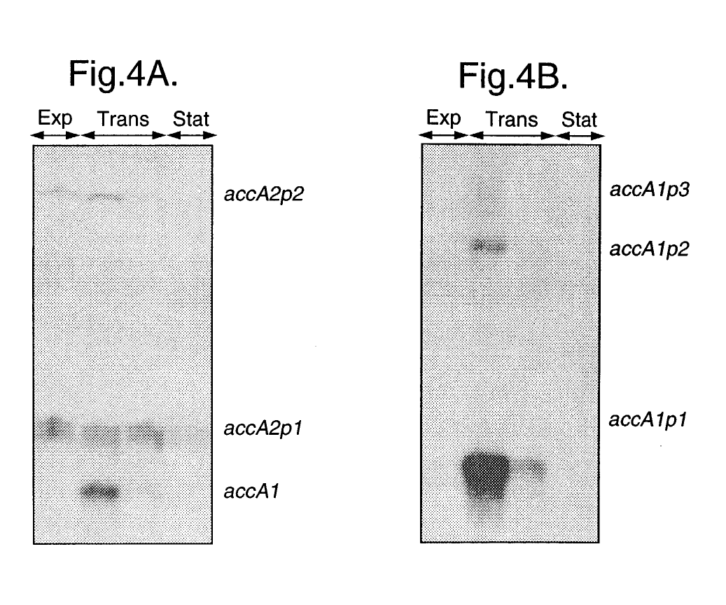

The levels of accA2 and accA1 mRNA present throughout growth were also studied by S1 protection experiments (FIG. 4). The probe used for accA2 was a 766 bp DNA fragment generated by PCR and uniquely labelled on the 5′end of the oligo corresponding to the sequence within accA2. This experiment showed the existence of three mRNA-protected fragments. The growth phase-dependent expression of two of them, accA2p1 and accA2p2, resemble very much that of the accBE operon. Thus, a constant and high level of expression occurs during exponential and transition phase (TP), while the transcription shuts down when the cultures reach stationary phase (FIG. 4A).

Considering that the nucleotide sequences of accA1 and accA2 are identical from the first two nucleotides upstream of the most probable GTG translation start sites down to the end of the probe (Rodríguez and Gramajo, 1999), it is important to note that a fragment of 185 bp of the accA2 probe could also be protected by the accA1 mRNA. Since the lowest RNA-protected fragment observed in FIG. 4A shows a different pattern of expression with respect to accA2p1 and p2, and considering that the size of the band corresponds to a 185 bp fragment, we believe that this band might represent the level of expression of accA1 (although we cannot rule out the existence of a third promoter for accA2, regulated in a different manner).

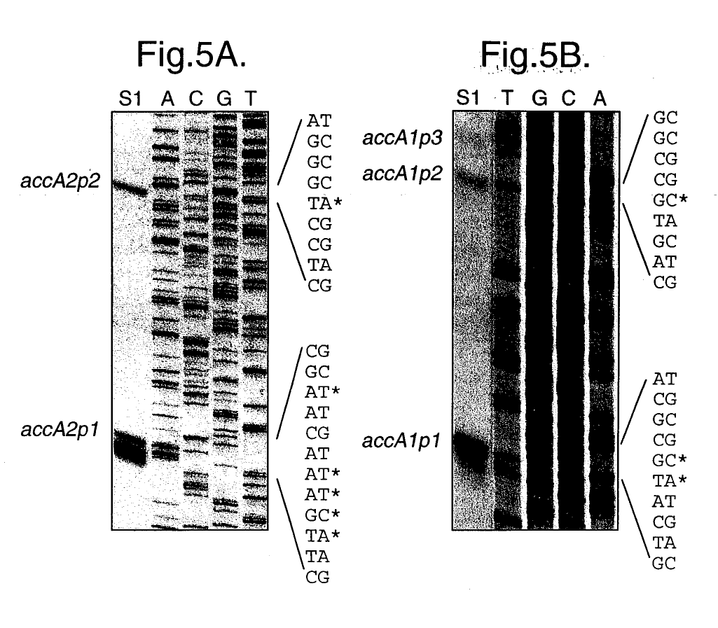

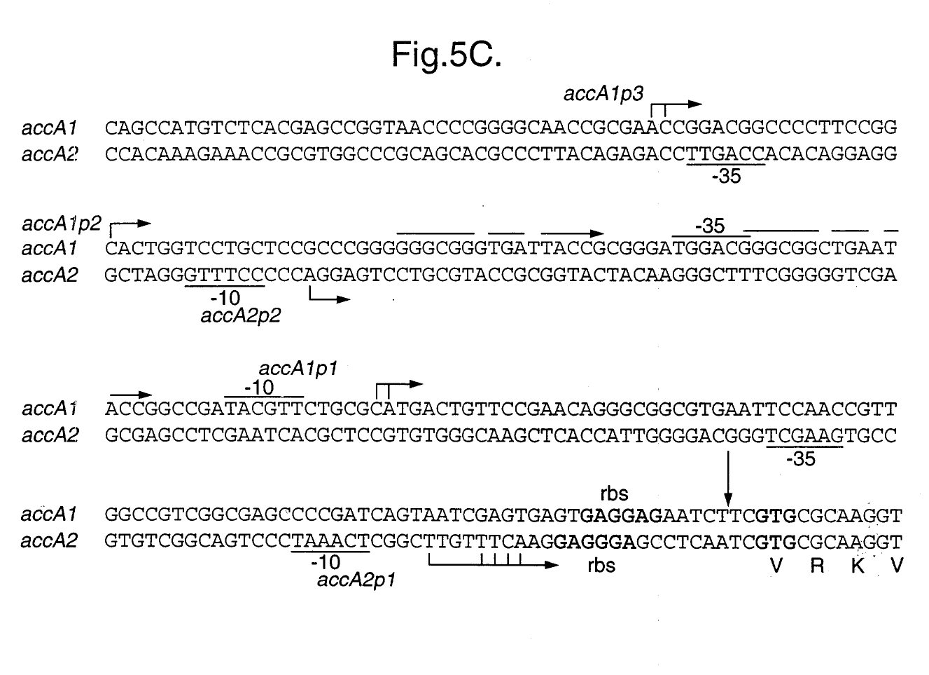

S1 nuclease protection of accA1 mRNA was performed by using a 563 bp PCR product, uniquely labelled at the 5′end of the downstream oligo, corresponding to a sequence within accA1. As shown in FIG. 4B, the expression of this gene occurs from at least three different putative promoters, and all of them showed a clear burst of expression during the first hours of the TP, which rapidly shut down during late TP. This pattern of transcription resembled very much the one observed for the third RNA-protected band found for accA2. The transcription starts sites for the accA2p1 and p2 were mapped by high resolution S1 mapping (FIGS. 5A and B). The transcription start points and the putative -10 and -35 promoter regions of these two promoters are shown in FIG. 5C. A certain degree of homology was found between the -10 consensus sequence of accA2p1 and p2 and the promoters recognised by the vegetative σ^{hrdB} (Strohl, 1992). High resolution S1 mapping of accA1 revealed that the transcription start point of the most abundant mRNA species starts 88 bp upstream of the GTG initiation codon of AccA1 and the putative -10 regions resemble, in some extent, the consensus sequences of promoters recognised by σ^{hrdB}. Interestingly, two direct repeat (DR) sequences of 16 bp, containing only two mismatches, were found flanking the putative -35 region of accA1p1 and the transcription start point of accA1p2 (FIG. 5C). These DRs could represent DNA binding sites recognised by a putative regulator. A third putative promoter, accA1p3, was also detected in longer exposures and the most probable nucleotide start sites are also indicated in FIG. 5C.

### EXAMPLE 5

### AccBE Genes are Essential in the Presence of Malonate

The presence of MatC and MatB homologues in S. coelicolor suggested that this micro-organism was potentially capable of transporting malonate within the cell through the MatC transporter, and then activating malonate to malonyl-CoA with the putative malonyl-CoA synthetase MatB. To test whether S. coelicolor was able to utilize malonate as a sole carbon and energy source, we grew S. coelicolor in a modified SMM medium with no casamino-acids and containing 0.4% malonate instead of glucose as a sole carbon source. In this medium S. coelicolor M145 was able to grow, indicating that MatC and MatB could be the proteins involved in the transport and activation of malonate to malonyl-CoA, and suggesting that a decarboxylase that could convert malonyl- into acetyl-CoA should also be present in this bacterium, to allow the use of malonate as a carbon and energy source.

This result encouraged us to test whether this route could also be an alternative pathway to provide malonyl-CoA to the cell. To prove this hypothesis we tried to obtain an acyl-CoA carboxylase minus mutant in the presence of malonate. For this we took spores of strain T124 and grew them in liquid MM containing 0.4% of malonate instead of glucose. After 36 h of growth we sonicated the mycelia and spread them in SFM medium containing 0.4% of malonate and incubated until sporulation. Spores were collected and treated in the same way one more time. Finally, spores harvested after the second round of sporulation were diluted out, inoculated in SFM malonate to give aprox. 500 colonies per plate and replica plated in SFM medium with or without Th. After analyzing approximately 5000 isolated colonies, no Th^{S} were obtained. This result indicates that although malonate can be efficiently used as a sole carbon and energy source, the pathway involved in its catabolism can not fulfill the malonyl-CoA requirements of the cell.

### EXAMPLE 6

### Construction of a Strain with the AccBE Operon Under the Control of a tipA Promoter

As shown above, the accBE operon, which encodes the carboxyl-transferase and a previously unidentified ε sub-unit of an acyl-CoA carboxylase, is essential for the viability of S. coelicolor A3(2). In order to regulate the expression of this operon and study its effect on the physiology of this microorganism, we constructed a conditional mutant strain where the expression of the accBE operon was under the control of the thiostrepton-inducible tipA promoter (Murakami, et al., 1989).

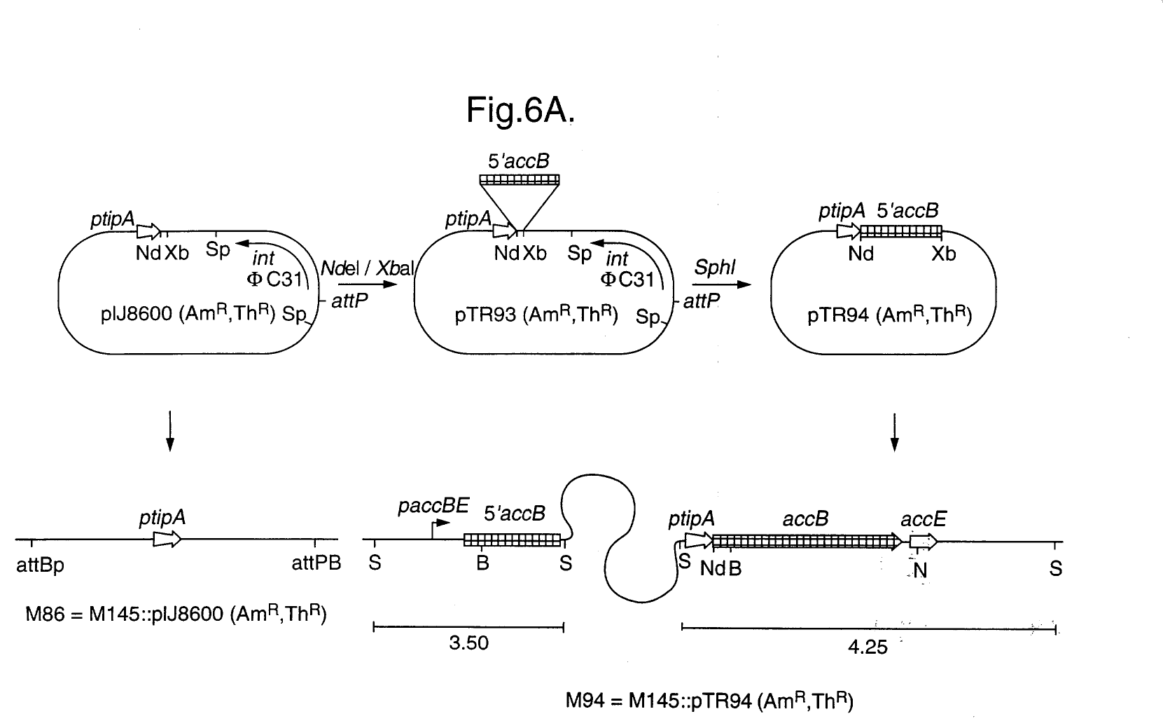

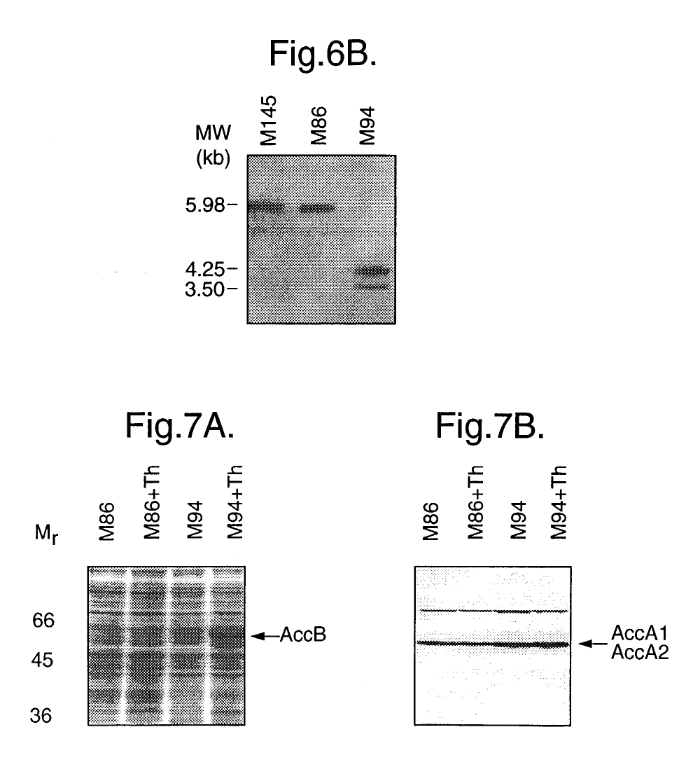

A 947 bp fragment containing a modified 5′end of the accB gene was cloned under the tipA promoter in pIJ8600 (Sun et al (1999) supra) to yield pTR93. After removal of the ΦC31 integration components (att and int) present in pTR93 we obtained pTR94, which was transformed into the E. coli strain ET12567/pUZ8002 (MacNeil et al (1992)/Paget et al (1999)). Conjugation of pTR94 into the S. coelicolor strain M145 gave several exconjugants Th^{R}. One of these exconjugants, designated M94, was purified in SFM medium for further analysis. Integration of pTR94 could only take place by Campbell recombination through the accBE homologous sequences, and this event should leave a complete copy of the accBE operon under the tipA promoter (FIG. 6A). To confirm that this event had occurred in M94, we performed Southern blot experiments of DNA samples prepared from strains M145, M94 and M86. The last strain (M86) was obtained by integration of pIJ8600 in the ΦC31 att site of the chromosome and used as the best isogenic control for M94 (FIG. 6A). As shown in FIG. 6B, a SacI digested DNA from M145 and M86 lights up a unique hybridisation band of 5.94 kb that contains the accBE operon. DNA from M94, instead, lights up two hybridising bands corresponding to the expected sizes for the integration of pTR94 in the accBE operon (FIGS. 6A and B).

### EXAMPLE 7

### Acyl-CoA Carboxylase Levels in M94 and M86

Cultures of the conditional accBE mutant M94 grew normally in YEME medium containing 5 μg of Th. Interestingly, in the absence of the antibiotic, the cultures were still able to grow, although at much lower rate. This experiment re-confirms the leakiness of the tipA promoter (M. J. Bibb, personal communication). In order to determine the levels of the acyl-CoA carboxylase in conditions of induction or non-induction we carried out the following protocol. YEME medium containing 10 μg of Am was inoculated with spores of M94 (or M86) to give and initial OD₄₅₀=0.1. Cultures were grown for 12 h at 30° C. and after that time 5 μg of Th was added to a half of each culture, keeping the other half as a control. Both flasks were then incubated for additional 24 h at 30° C. The harvested mycelia were disrupted by sonication and cell debris removed by centrifugation. Cell-free extracts were finally analysed by SDS-PAGE and used for enzyme assays. FIG. 7A shows a 60 kDa protein that is only induced in cultures of M94 grown in the presence of Th; the size of this protein corresponded to the molecular mass of AccB. We were not able to detect an inducible band corresponding to AccE. The levels of the biotinylated components (AccA1 or AccA2) of the acyl-CoA carboxylase, in each of the cell-free extracts, were analysed by a modified Western Blotting procedure (FIG. 7B). As shown in this figure the levels of AccA1 and/or AccA2 were not modified by presence of Th. However, cell free-extracts of M94 do contain a slightly higher amount of the 65 kDa protein compared to M86.

ACCase and PCCase activities were assayed in cell-free extracts of M94 and M86. The levels of both enzyme activities were similar in cell-free extracts prepared from cultures of M86 grown in the presence or in the absence of Th (Table 3). Cell-free extracts prepared from induced cultures of M94 show instead a remarkable increase in both ACCase (11.5 fold) and PCCase (3.5 fold) activities, compared with the levels found in non-induced cultures of the same strain or in M86. Moreover, if the enzyme levels found in the wild type strain M145 (Rodríguez and Gramajo, 1999) are compared with those found for M94, the increase in ACCase and PCCase levels were still 4- and 2-fold, respectively (Table 3). These results indicate that by overproducing only two (β and ε) of the three sub-units that form the acyl-CoA carboxylase of S. coelicolor we can increase significantly the levels of this enzyme activity.

### EXAMPLE 8

### Influence of the Acyl-CoA Carboxylase Levels in the Physiological Properties of M94

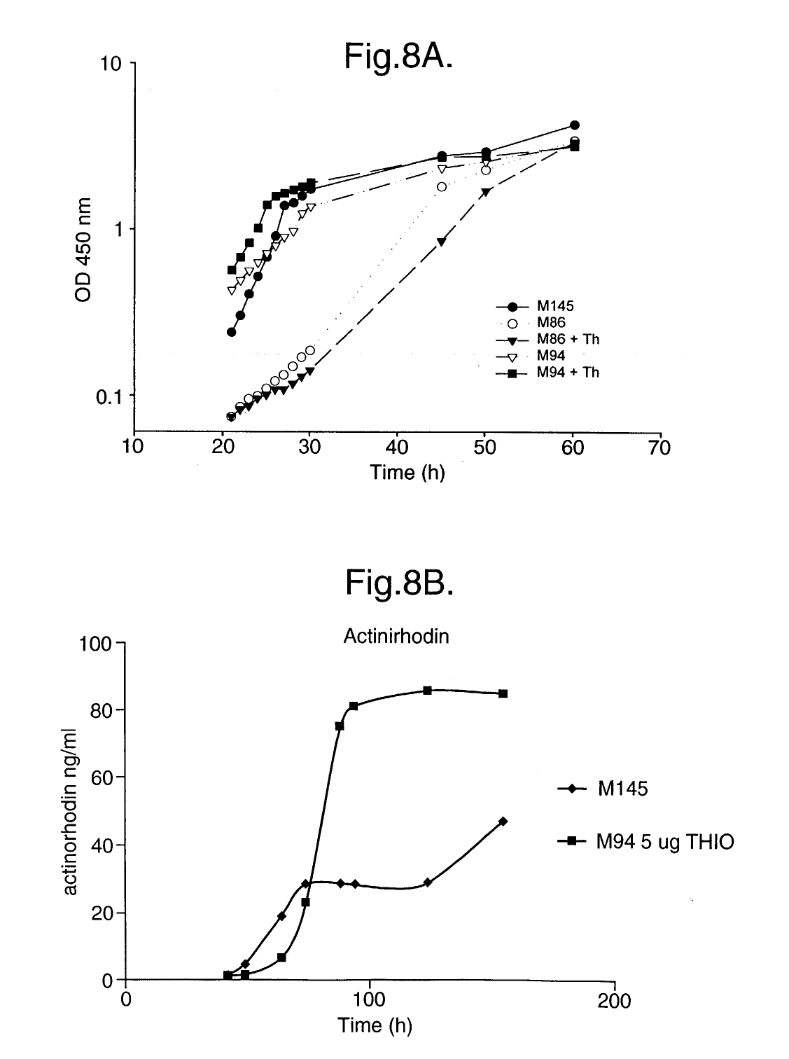

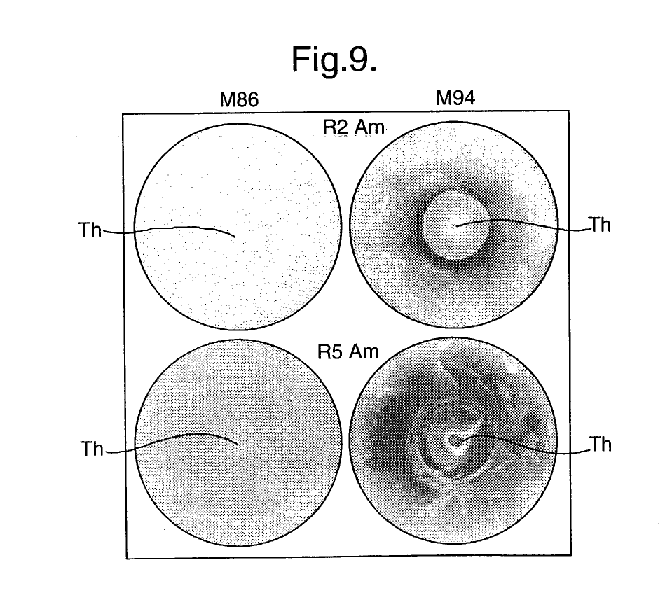

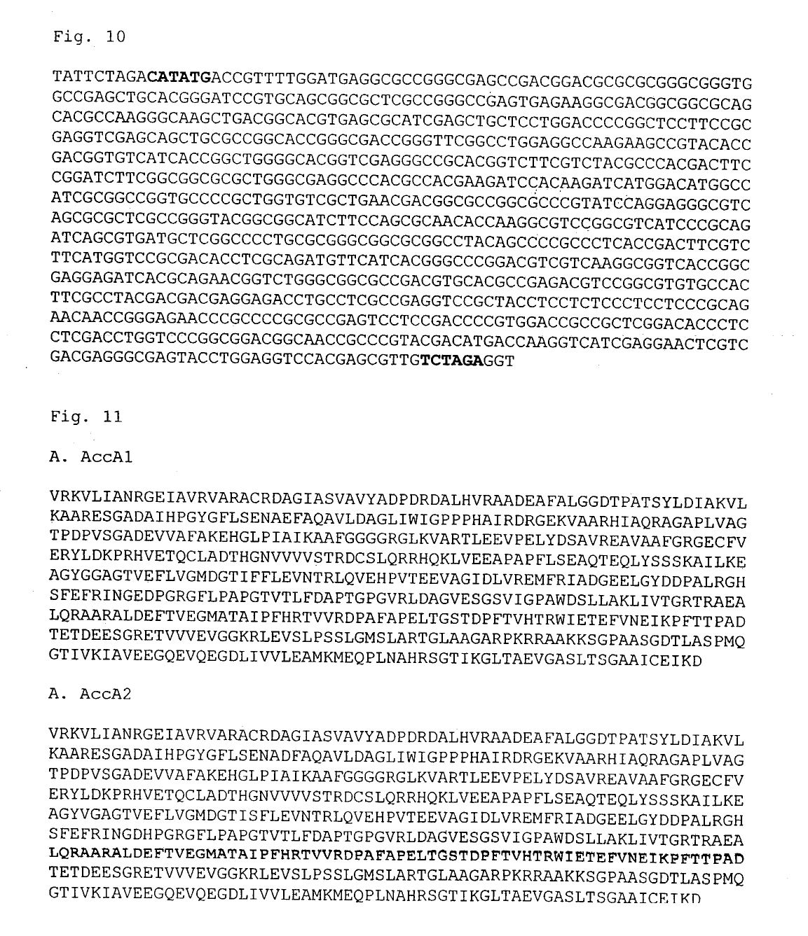

Growth curves (FIG. 8A) were determined for the conditional mutant M94 and for M86 by inoculating a spore suspension in YEME medium supplemented with 10 μg of Am, with or without the addition of 5 μg of Th. For M145, YEME medium without the addition of any antibiotic was used. M94 supplemented with the inducer (Th) showed a growth rate during exponential phase very similar to M145, judged from the slope of the curves. However, the initiation of growth for M94 seems to occur sooner than in M145, reaching the stationary phase earlier than the wild type strain. When the cultures were not supplemented with Th, M94 grew considerably slower, reaching stationary phase several hours latter than in the presence of Th. Also, the final OD reached by M94 in the presence of Th and by M145 were very similar (OD₄₅₀=3) after 60 h of growth. Cultures of M86 grew very slowly compared with M94 and M145, independently of the presence or not of Th. However, these cultures levelled off at the final OD reached by M145 and M94 after 50 h of growth.

Actinorhodin and undecylprodigiosin were also quantitated throughout growth. Table 4 shows that antibiotic production was only detected in cultures of M94 grown in the presence of 1 or 5 μg of Th. No antibiotic production was observed in cultures of M145 or M94 without Th, at least until after 60 h of growth. No antibiotic production was detected in M86.

To determine the effect of Th induction in M86 and M94, 1 μg of the antibiotic was spotted to a confluent lawn of these strains in R2 and R5 medium supplemented with 10 μg of Am. A striking stimulatory effect in both sporulation and antibiotic production was observed in M94 after 48 h. No stimulation of growth or antibiotic production was observed in M86.

FIG. 8B shows the stimulatory effect on actinorhodin production in M94 compared to M145 in cultures grown in the presence of 5 μg of Th.

### EXAMPLE 9

### Co-Expression of AccA, AccB and AccE in S. coelicolor

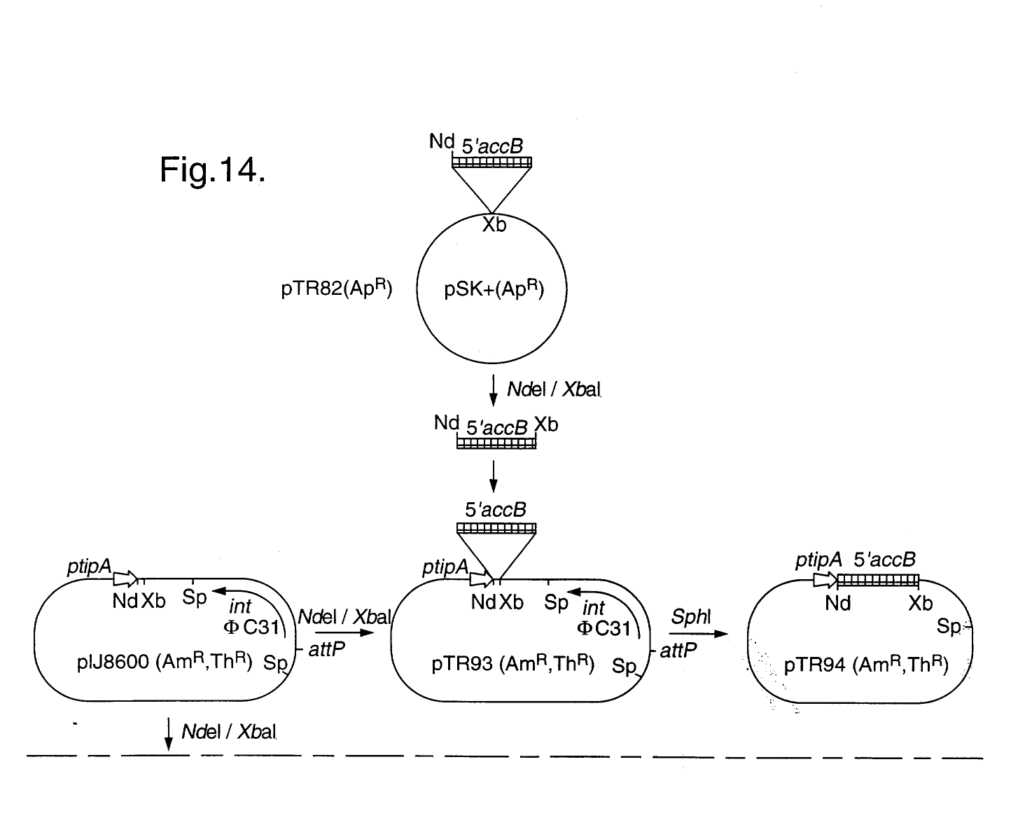

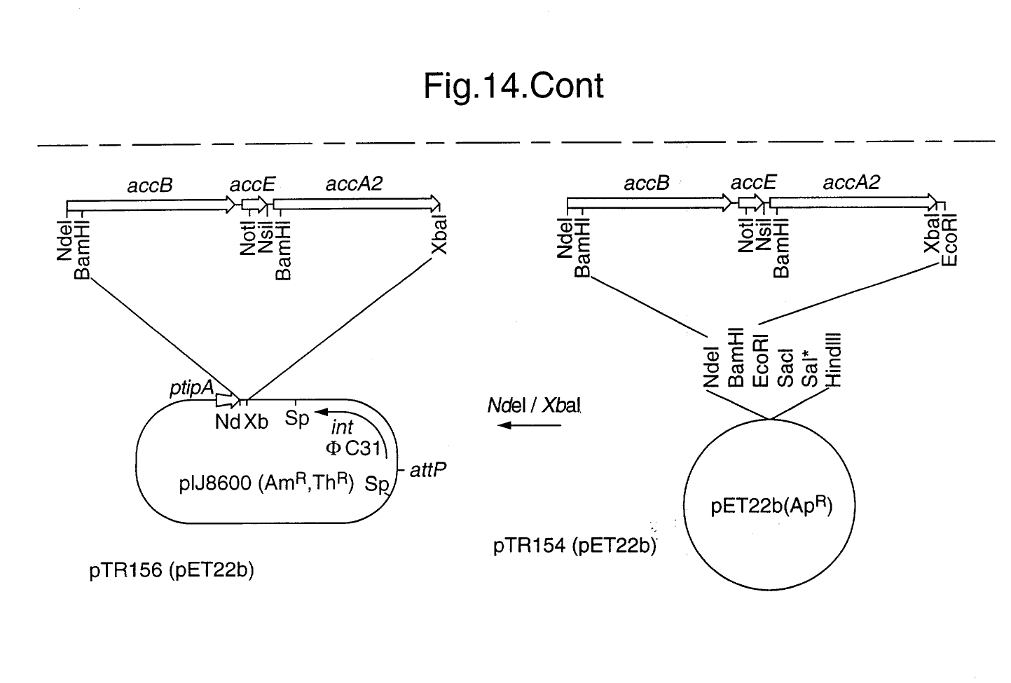

The NdeI-XbaI fragment of pTR154 (FIG. 14) is introduced into pIJ8600 and then transformed into S. coelicolor M145 (FIG. 14). Transformants are selected with apramycin and thiostrepton. Overexpression of the three components accA2, accB and accE results in increased. ACCase activity and antibiotic production compared to the wild type M145 strain.

### DISCUSSION

The use of pccB (Rodríguez and Gramajo, 1999) as an heterologous probe, allowed the successful isolation of a chromosomal DNA fragment containing accB, a gene encoding for a putative new carboxyl transferase of S. coelicolor. This predicted function was based on the high percentage of identity that AccB showed not only to the S. coelicolor PccB, but to several others biochemical and/or genetically characterized carboxyl transferases reported for actinomycetes, such as the PccB of Sac. erythraea (Donadio, et al., 1996) and to a less extent to the AccD5 of M. tuberculosis (Cole, et al., 1998) and PccB of M. leprae (Doukhan, 1995). An interesting finding from the analysis of the cloned sequence was the presence of a very small ORF, named accE, immediately downstream of accB.

The successful expression of accB, accE and the BC-BCCP-(biotin carboxylase- and biotin carboxylase carrier protein-)encoding genes accA1 and accA2 in E. coli allowed in vitro studies to be performed in order to understand the role of the corresponding encoded proteins as components of a previously uncharacterized acyl-CoA carboxylase. The reconstitution, by mixing cell-free extracts of E. coli containing AccB and AccA1 (or AccA2), of an active enzyme with the ability to carboxylate either acetyl- or propionyl-CoA clearly established that AccB was the carboxyl transferase component of an acyl-CoA carboxylase complex. Interestingly, the small polypeptide, AccE, also showed to play an important role in the reconstitution of a fully active enzyme complex (Table 2). It remains to be elucidated whether this protein plays a role as an allosteric regulator of the enzyme or whether it is a structural component of the complex. Thus, our results represent the first characterization, at both the genetic and biochemical levels, of a prokaryotic acyl-CoA carboxylase.

All the acyl-CoA carboxylases studied so far contain the three functional domains in two individual polypeptides (for a review see Brownsey et al., 1997 ), and none of the purified complexes have shown the presence of a small component equivalent to AccE. Therefore, this might be a distinctive feature for Streptomyces sp. In addition, no AccE homologues have been found in any of the bacteria genomes sequenced so far, an observation that could also support this hypothesis.

Malonyl-CoA is an essential component of all living organisms, since it is the main elongation unit for fatty acid biosynthesis (Brownsey et al., 1997). This primary metabolite is synthesised in most species through the carboxylation of acetyl-CoA by an ACCase (Bloch and Vance, 1977). If this was also the case for S. coelicolor and, if AccB was the component of an essential acyl-CoA carboxylase, mutation of this gene should be lethal for the micro-organism. Replacement of the wild-type accB for the Hyg^{R} mutant allele prove to be unsuccessful, and it only occurred when a second copy of the accBE genes was present in the chromosome (FIG. 2B).

These experiments clearly indicated that at least accB was essential for S. coelicolor viability. The fact that both AccA2 (Rodríguez and Gramajo, 1999) and AccB have proved to be essential, along with the fact that acyl-CoA carboxylase reconstituted in vitro with these two sub-units has the ability to recognise either acetyl- or propionyl-CoA as substrates, strongly suggests that AccA2 and AccB are the α and β components of an essential acyl-CoA carboxylase, whose main physiological role should be the biosynthesis of malonyl-CoA. The transcriptional levels of accB and accA2 throughout growth (FIGS. 3A and 4A) also support this interpretation, since both genes are principally transcribed during exponential and transition phase. Moreover, ACCase and PCCase activities also showed the highest and constant levels of activities during exponential and transition phase while in stationary phase the activities were low but readily measurable.

In S. coelicolor, besides the obvious need for malonyl-CoA biosynthesis during vegetative growth, there is also a requirement for this metabolite during transition and stationary phase, since at least two secondary metabolites (undecylprodigiosin and actinorhodin) are synthesised during these growth-phases and they both require malonyl-CoA for their biosynthesis. Hence, if the ACCase is the only enzyme that synthesises malonyl-CoA in this bacterium, its presence will be also required during the idiophase.

According to the proposed composition of this enzyme complex and based on the transcriptional studies, we propose that the low level of expression of accA2 and accBE during stationary phase is sufficient to produce enough of the α and β components for an active acyl-CoA carboxylase. From the observation that accA1 mRNA peaks during transition phase, we propose that enough AccA1 might be present in the cytoplasm to compete with AccA2 as the main α sub-unit of this enzyme complex in the stationary phase. However, no difference in antibiotic production has been found between M145 and the isogenic accA1 mutant MA4 (Rodríguez and Gramajo, 1999).

We have clearly demonstrated the ability of S. coelicolor to efficiently utilize malonate as a sole carbon and energy source. A putative pathway for the utilization of this substrate could involve the R. trifolii MatC and MatB homologues which are found in the genome of S. coelicolor. The biochemical characterization of MatB in R. trifolii demonstrated that this protein is a malonyl-CoA synthetase, which catalyzes the formation of malonyl-CoA directly from malonate and CoA. MatC, instead, has not been characterized biochemically but computer analysis indicate that it is a transmembrane protein that could function as a dicarboxylate (malonate for example) carrier (An and Kim, 1998). If these enzymes were part of the pathway that allows S. coelicolor to utilize malonate as a sole carbon source, one could also presume that the malonyl-CoA synthesized by MatB should fulfill the malonyl-CoA requirements of the micro-organism. However, we could not show that under these conditions the essential acyl-CoA carboxylase becomes dispensable.

Interestingly, the addition of 0.4% malonate to SFM and glucose-MM media produced a clear stimulation of actinorhodin production (data not shown). From this we propose that higher levels of malonyl-CoA were probably available under this growth conditions. From this, and the observation that even the limited levels of the ACCase activity found during the stationary phase of growth of this bacterium are sufficient to allowed regular levels of antibiotic production, the inventors propose that increasing the expression of the ACCase components will probably lead to an improved production of antibiotics.

### EXPERIMENTAL PROCEDURES

### Bacterial Strains, Cultures and Transformation Conditions

S. coelicolor A3 (2) strain M145 (SCP1⁻ SCP2⁻) was manipulated as described by Hopwood et al. (1985). The strain was grown on various agar media—SFM (Rodríguez and Gramajo, 1999), R2 and R5—or in 50 ml SMM or YEME liquid media (Hopwood et al (1985) supra). Escherichia coli strain DH5α (Hanahan 1983) was used for routine subcloning and was transformed according to Sambrook et al. (1989). Transformants were selected on media supplemented with the appropiate antibiotics : ampicillin (Ap) 100 μg/ml; apramaycin (Am) 100 μg/ml; chloramphenicol (Cm) 25 μg/ml or kanamycin (Km) 30 μg/ml. Strain BL21(DE3) is an E. coli B strain [F⁻ ompT (r_{B}⁻ m_{B}⁻) (DE3)] lysogenized with 1DE3, a prophage that expresses the T7 RNA polymerase downstream of the IPTG-inducible lacUV5 promoter (Studier & Moffat, 1986). ET12567/pUZ8002 (MacNeil et al (1992)/Paget et al (1999)) was used for E. coli-S. coelicolor conjugation experiments (Bierman, 1992). For selection of Streptomyces transformants and exconjugants, media were overlayed with thiostrepton (Th) (300 μg per plate), hygromycin (Hyg) (1 mg per plate) or apramycin (Am) (1 mg per plate). Strains and recombinant plasmids are listed in Table 1.

### Growth Conditions, Protein Expression and Preparation of Cell-Free Extracts

S. coelicolor M145 was grown at 30° C. in shake flasks in YEME medium for 24-48 h. When necessary, 10 mg Am ml⁻¹ or 5 mg Th ml⁻¹ were added to the medium. Mycelia were harvested by centrifugation at 5000×g for 10 min at 4° C., washed in 100 mM potassium phosphate buffer pH 8 containing 0.1 mM DTT, 1 mM EDTA, 1 mM PMSF and 10% glycerol (buffer A) and resuspended in 1 ml of the same buffer. The cells were disrupted by sonic treatment (4 or 5 s bursts) using a VibraCell Ultrasonic Processor (Sonics & Materials, Inc.).

Cell debris was removed by centrifugation and the supernatant used as cell-free extract. For the expression of heterologous proteins, E. coli strain BL21(DE3) harbouring the appropriate plasmids were grown at 37° C. in shake flasks in LB medium in the presence of 25 μg Cm ml⁻¹ or 100 μg Ap ml⁻¹ for plasmid maintenance. For the expression of biotinylated proteins, 10 μM d-biotin was supplemented to the medium. Overnight cultures were diluted 1:10 in fresh medium and grown to A₆₀₀ 0.4-0.5 before the addition of IPTG to a final concentration of 0.1 mM. Induction was allowed to proceed for 4 h. The cells were then harvested, washed and resuspended in 1 ml buffer A. Cell-free extracts were prepared as described above.

### Protein Methods

Cell-free extracts were analysed by denaturing (SDS)-PAGE (Laemmli, 1970) using the Bio Rad mini-gel apparatus. The final acrylamide monomer concentration was 12% (w/v) for the separating gel and 5% for the stacking gel. Coomassie brilliant blue was used to stain protein bands. The biotinylated proteins were detected by a modification of the Western blotting procedure described by Nikolau et al. (1985). After electrophoretic separation, proteins were electro-blotted onto nitrocellulose membranes (Bio-Rad) and probed with alkaline phosphatase-streptavidin conjugate (Bio-Rad) diluted 1:10000. Protein content was determined by the method of Bradford (1976) with BSA as standard.

### In Vitro Reconstitution and Assay of the Acyl-CoA Carboxylase Complex

In vitro reconstitution of the enzyme complex was carried out by mixing 100 μg of each of the cell-free extracts shown in Table 2 in a final volume of 300 μl. When AccE was not included in the incubation mix, 100 μg of BSA were added instead. The mixes were incubated for 1 h at 4° C. and 100 μg of each used for enzyme assay.

ACCase and PCCase activities in cell-free extracts were measured following the incorporation of H¹⁴CO₃⁻ into acid non-volatile material (Huanaiti & Kolattukudy, 1982; Bramwell et al., 1996). The reaction mixture contained 100 mM potassium phosphate pH 8.0, 300 μg BSA, 3 mM ATP, 5 mM MgCl₂, 50 mM NaH¹⁴CO₃ [specific activity 200 μCi mmol⁻¹ (740 kBq mmol⁻¹)], 1 mM substrate (acetyl-CoA or propionyl-CoA) and 100 μg cell-free protein extract in a total reaction volume of 100 μl. The reaction was initiated by the addition of NaH¹⁴CO₃, allowed to proceed at 30° C. for 15 min and stopped with 200 μl 6 M HCl. The contents of the tubes were then evaporated to dryness at 95° C. The residue was resuspended in 100 μl water, 1 ml of Optiphase liquid scintillation (Wallac Oy) was added and ¹⁴C radioactivity determined in a Beckman scintillation liquid counter. Non-specific CO₂ fixation by crude extracts was assayed in the absence of substrate. One unit of enzyme activity catalysed the incorporation of 1 μmol ¹⁴C into acid-stable products per min.

### DNA Manipulations

Isolation of chromosomal and plasmid DNA, restriction enzyme digestion and agarose gel electrophoresis were carried out by conventional methods (Sambrook et al., 1989; Hopwood et al., 1985). Southern analyses were performed by using P-labelled probes made by random oligonucleotide priming (Prime-a-gene kit; Promega).

### Gene Cloning and Plasmid Construction

The synthetic oligonucleotides TC1, 5′-CAGAATTCAAGCAGCACGCCAAGGGC AAG, and TC2, 5′-CAGAATTCGATGCCGTCGTGCTCCTGGTC, were used to amplify an internal fragment of the S. coelicolor pccB gene. The reaction mixture contained 10 mM Tris-HCl pH 8.3, 50 mM KCl, 1 mM MgCl₂, 6% glycerol, 25 μM of each of the four dNTPs, 2.5 U Taq DNApolymerase, 20 pmol of each primer and 50 ng of S. coelicolor chromosomal DNA in a final volume of 100 μl. Samples were subjected to 30 cycles of denaturation (95° C., 30 s), annealing (65° C., 30 s) and extension (72° C., 1 min). A 1 kb PCR fragment was used as a ³²P-labelled probe to screen a size-enriched library. A 2.7 kb BamHI fragment containing an incomplete accB gene was cloned in BamHI-cleaved pBluescript SK(+), yielding pTR62.

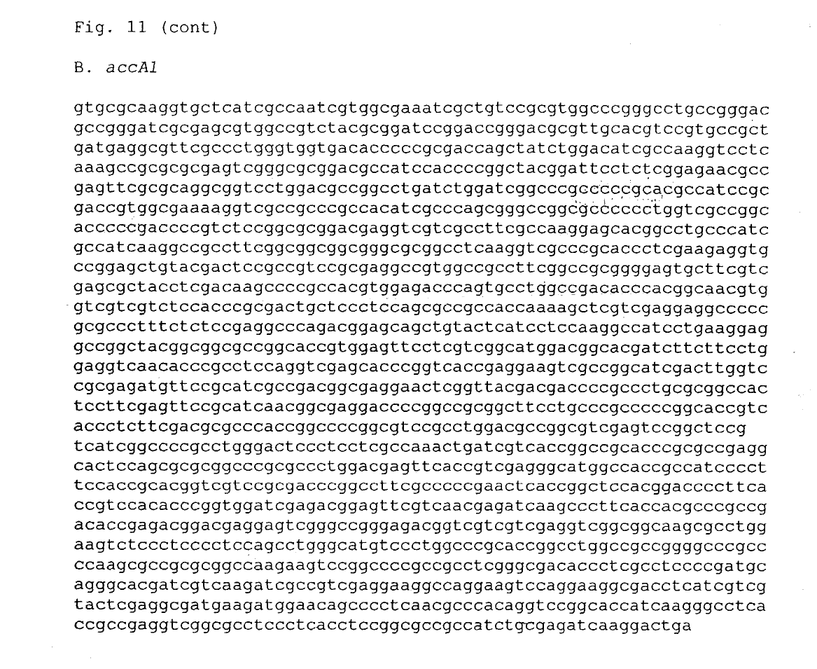

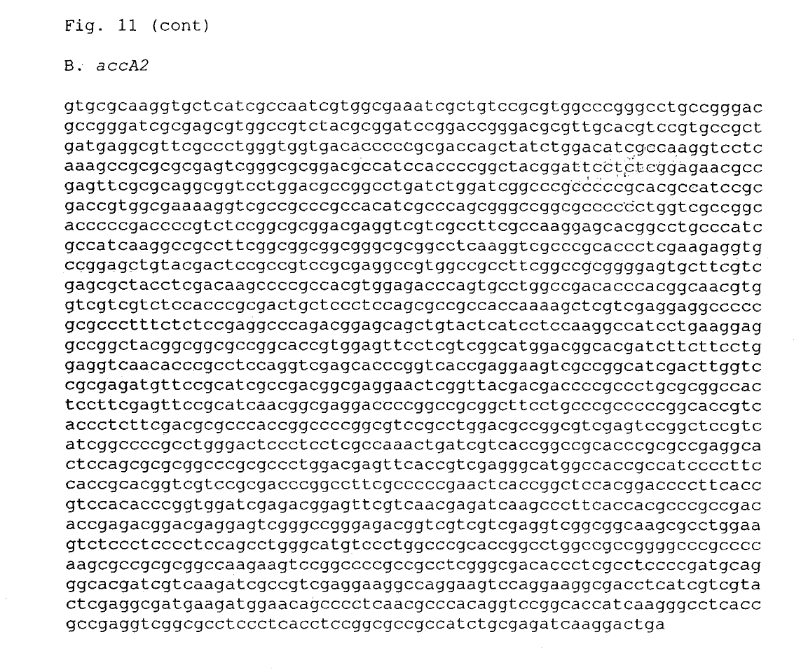

The synthetic oligonucleotide TC16 (5′-TATTCTAGACATATGACCGTTTTGGATGAGG, used to introduce an NdeI site at the translational start codon of the S. coelicolor accB gene) and TC17 (5′-ACCTCTAGACAACGCTCGTGGACC, used to introduce an XbaI site in the accB coding sequence) were used to amplify an internal fragment of S. coelicolor accB gene, having the sequence shown in FIG. 10. The reaction mixture was the same as the one indicated above. Samples were subjected to 30 or 35 cycles of denaturation (95° C., 30 s), annealing (65° C., 30 s) and extension (72° C., 1 min). The 1 kb PCR product was digested with NdeI and XbaI (these sites were introduced in the 5′ ends of the oligos TC16 and TC17 and are shown in bold in FIG. 10) and cloned in XbaI-cleaved pBluescript SK(+) in E. coli DH5α, yielding pTR82. This plasmid was digested with BstEII and SacI, ligated with a BstEII-SacI fragment cleaved from pRM08 and introduced by transformation into E. coli DH5α, yielding pTR87.

An NdeI-XbaI fragment from the plasmid pTR82 was cloned in NdeI-XbaI-cleaved pIJ8600 (Sun et al (1999)), yielding pTR93. In order to place the chromosomal copy of accBE operon under the tipA promoter we removed from pTR93 a HindIII fragment containing the int gene and att of ΦC31, yielding pTR94. Plasmid pTR94 was transformed into strain ET12567/pUZ8002 and transferred by conjugation to S. coelicolor M145 (Hopwood et al (1985)).

A NdeI-SacI fragment from the plasmid pTR87 was cloned in NdeI-SacI-cleaved pET22b(+) (Novagen) (pTR88), thus placing the accBE operon under the control of the powerful T7 promoter and ribosome-binding sequences. The synthetic oligonucleotides NaccE, 5′-TTATCTAGACATATGTCCCCTGCCGAC, used to introduce an NdeI site at the translational start codon of the S. coelicolor accE gene, and CaccE, 5′-ATGAATTCTATGCATCGGGTCAGCGCCAGCTG, were used to amplify the accE gene of S. coelicolor. The reaction mixture was the same as the one indicated above. Samples were subjected to 35 cycles of denaturation (95° C., 30 s), annealing (65° C., 30 s) and extension (72° C., 30 s). The PCR product was cloned using pGEM-T easy vector (Promega) in E. coli DH5α, yielding pTR106. A NdeI-EcoRI fragment from the plasmid pTR106 was cloned in NdeI-EcoRI-cleaved pET22(b) (Novagen) yielding the plasmid pTR107, thus placing the accE gene under the control of the powerful T7 promoter and ribosome-binding sequences.

Plasmid pIJ8600 was digested with BglII and EcoRI and the fragment containing oriT RK2, ori pUC18, attP site, int ΦC31 and aac(3)IV (Am^{R} cassette) genes was ligated with a linker containing the following enzymes (Mike Butler personal comunication): BglII, AseI, EcoRI, BglII, NdeI, KpnI, XbaI, PstI, HindIII, BamHI, SstI, NotI and EcoRI, yielding pTR141. A 4.0 kb KpnI fragment containing the complete accBE operon from pRM08 was cloned into KpnI-cleaved pTR141, yielding pTR149.

For an efficient over-expression in S. coelicolor of the three components of the acyl-CoA carboxylase complex of this micro-organism, we carried out the construction of pTR156 through the following steps. First we did a PCR amplification of the chromosomal accBE operon using the oligo TC16 (5′-TATTCTAGACATATGACCGTTTTGGATGAGG 3′), to introduce a NdeI site at the translation start codon of accB, and the oligo C-accE (5′ATG AAT TCT ATG CAT CGG GTC AGC GCC AGC 3′) to introduce a NsiI restriction site at the 3′ end of accE. The amplified DNA, was then cloned into pGEM-T (Promega), to give pTR99. To introduce a NsiI site upstream of the RBS of accA2 we amplified this gene using the oligo N-accA2 (5′ ATG AAT TCA TGC ATG AGG GAG CCT CAA TCG 3′), for the 5′ end and the oligo C-accA2 (5′ AGA TCT AGA TCA GTC CTT GAT CTC GC 3′) containing a XbaI and a EcoRI site, for the 3′ end of the gene. The amplified DNA was cloned in pGEM-T to give pTR112. The NdeI-NsiI DNA fragment from pTR99 and the NsiI-EcoRI fragment isolated from pTR112 were finally cloned into pET22(b) (Stratagene), previously digested with NdeI and EcoRI, to yield pTR154. In order to introduce these genes in S. coelicolor we sub-cloned the NdeI-XbaI fragment, containing accBE and accA2, from pTR154 to pIJ8600 digested with the same enzymes to give pTR156. See FIG. 14 for plasmid constructions.

### Nucleotide Sequencing

The sequence of the SphI original fragment was performed from plasmids DNA constructed by subcloning ApaI DNA fragments from pRM08 into pSKBluescribe SK(+). Synthetic oligonucleotides were used to complete the sequence. The nucleotide sequence of the accBE region was determined by dideoxy sequencing (Sanger et al., 1977) using the Promega TaqTrack sequencing kit and double-stranded DNA templates. The complete sequence of the 1C2 cosmid, that includes the SphI fragment harbouring accBE, is available from the S. coelicolor genome sequencing project.

### S1 Nuclease Mapping

For each S1 nuclease reaction, 30 μg of RNA were hybridized in NaTCA buffer (Murray, 1986); solid NaTCA (Aldrich) was dissolved to 3M in 50 mM PIPES (pH 7.0), 5 mM EDTA, to about 0.002 pmol (approximately 10⁴ cpm) of the following probes. For accA2 the synthetic oligonucleotide 5′-GCTTTGAGGACCTTGGCGATG (accA2down), corresponding to the sequence within the coding region of accA2, was uniquely labelled at the 5′ end of the oligonucleotide with [³²P]-ATP using T4 polynucleotide kinase. The labelled oligo was then used in the PCR reaction with the unlabelled oligonucleotide (accA2up) 5′-GAAGTACAGGCCGAAGACCAC, which corresponds to a region upstream of the accA2 promoter region, to generate a 766 bp probe. For accA1 the synthetic oligonucleotide (accA1down) 5′-GCGATTTCGCCACGATTGGCG, corresponding to the region within the coding region of accA1, was uniquely labelled with [³²P]-ATP using T4 polynucleotide kinase at the 5′ end of the oligonucleotide. The accA1down oligo was later used in the PCR reaction with the unlabelled oligonucleotide (accA1up) 5′-CCGATATCAGCCCCTGATGAC, which corresponds to a region upstream of the accA1 promoter to generate a 563 bp probe. For accB the synthetic oligonucleotide (accBdown) 5′-CGTCAGCTTGCCCTTGGCGTG, corresponding to the region within the coding region of accB, was uniquely labelled with [³²P]-ATP using T4 polynucleotide kinase at the 5′ end of the oligonucleotide. accBdown was then used in the PCR reaction with the unlabelled oligonucleotide (accBup) 5′-CTACGCTCCGGGTGAGCGAAC, which corresponds to a region upstream of the accB promoter, to generate a 483 bp probe. For accBE the synthetic oligonucleotide (accBEdown) 5′-GGAGGGCCGTGATGGCGGCGACTTCCTCGGG, corresponding to the region within the coding region of accE was uniquely labelled with [³²P]-ATP using T4 polynucleotide kinase at the 5′ end of the oligonucleotide. The accBEdown oligo was then used in the PCR reaction with the unlabelled oligonucleotide (accBEup) 5′-GAGGAACTGGTACGCGCGGGCG(GTACAAGCAAGCT), which corresponds to a region within the coding region of accB (bracketed oligonucleotides are a tail added to the probe to differentiate probe reannealing from fully protected DNA-RNA complexes), to generate a 563 bp probe. Subsequent steps were as described by Strauch et al. (1991).

### Determination of Actinorhodin

1 ml of whole broth was mixed with 0.5 ml of 3N KOH to give a final concentration of 1N KOH. The solutions were mixed vigorously and centrifuge at 4000×g for 5 minutes. The supernatant was collected and measured at A_{640 nm}. Actinorhodin concentration was calculated using the molar extinction coefficient (in 1 N KOH) at 640 nm of 25.320 (Bystrykh et al., 1996).

### Determination of Undecylprodigiosin

This was carried out according to the procedure of Hobbs et al. (1990).

### REFERENCES

An, J. H. et al. (1998) Eur J Biochem 257: 395-402.

Behal, V. et al. (1977) Phytochemistry 16: 347-350.

Bierman, M. et al. (1992) Gene 116: 43-49.

Bloch, K. and Vance, D. (1977) Annu Rev Biochem 46: 263-298.

Bradford, M. (1976) Anal Biochem 72:248.

Bramwell, H. et al. (1996) Microbiol 142: 649-655.

Bramwell, H. et al. (1993) Biochem J 293:131-136.

Brownsey, R. W. et al. (1997) Biochem Soc Trans 25: 1232-1238.

Buttner, M. J. et al. (1990) J Bacteriol 172: 3367-3378.

L. V. Bystrykh, M. A. et al. (1996). J Bacteriol. 187: 2238-2344.

Cole, S. T. et al. (1998) Nature 393: 537-544.

Donadio, S. et al. (1996) Mol Microbiol 19: 977-984.

Doukhan, L. et al. (1995) Gene 165: 67-70

Erfle, J. D. (1973) Biocim Biophys Acta 316: 143-155.

Gramajo, H. C. et al. (1993) Molecular Microbiology 7: 837-845.

Hanahan (1983) J. Mol. Biol. 166: 557-580.

Harwood, J. L. (1988) Annu Rev Plant Physiol Plant Mol Biol 39: 101-138.

Hasslacher, M. et al. (1993) J. Biol. Chem. 268: 10946-10952.

Henrikson, K. P. and Allen, S. H. G. (1979) J Biol Chem 254:5888-5891.

Hobbs, G. et al. (1990) J Gen Microbiol 136:2291-2296.

Hopwood, D. A. et al. (1985) Genetic manipulation of Streptomyces: A laboratory manual. John Innes Foundation, Norwich.

Hopwood, D. A. and Sherman, D. H. (1990) Ann Rev Genet 24:37-66.

Hunaiti, A. R. and Kolattukudy, P. E. (1982) Arch Biochem Biophys 216:362-371.

Laakel, M. et al. (1994) Microbiol 140: 1451-1456.

Laemmli, U. K. (1970) Nature 227:680-685.

Li, S. J. and Cronan. J. E., Jr. (1993) J Bacteriol 175: 332-340.

MacNeil et al. (1992) Gene 115: 119-125

Murakami, T. et al. (1989) J Bacteriol 171: 1459-1466.

Murray (1986) Anal Biochem 158(1): 165-170.

Nikolau, B. J. et al. (1981) Arch Biochem Biophys 211: 605-612.

Paget et al. (1999) Mol. Microbiol. 3: 97-107

Perez, C. A. et al. (1998) Microbiology 144: 895-903.

Pizer, E. S. et al. (1996) Cancer Res. 56: 2745-7.

Polakis, S. et al. (1972) J Biol Chem 247: 1335-1337.

Redenbach, M. et al. (1996) Mol Microbiol 21: 77-95.

Rodríguez, E. and Gramajo, H. (1999) Microbiology 145: 3109-3119.

Saggerson, E. D. et al. (1992) Adv Enzyme Regul 32: 285-306.

Sambrook, J. et al. (1989) Molecular Cloning: a Laboratory Manual. Cold Spring Harbor, N.Y.: Cold Spring Harbor Laboratory Press.

Sanger, F. et al. (1977) Proc Natl Acad Sci USA 74: 5463-5467.

Strauch, E. et al. (1991) Mol Microbiol 5: 289-298.

Strohl, W. R. (1992) Nucleic Acids Res. 20: 961-974.

Studier and Moffatt 1986 J Mol Biol 189(1): 113-30.

Sun et al. (1999) Microbiology 145: 2221-2227

Westrich, L. et al. (1999) FEMS Microbiol. Lett. 170: 381-387.

All the above references are hereby incorporated by reference in their entirety, individually and for all purposes.

TABLE 1
Strains and plasmids used.

| Strain/plasmids | Description | Reference/source |
|---|---|---|
| S. coelicolor | | |
| M145 | Parental strain SCP 1⁻ SGP2⁻ | Hopwood et al. |
| | | (1985) |
| T124 | M145 (accB:pTR124), Th^{R}, Hyg^{R} | This work |
| T149 | T124 containing pTR149 integrated in the att site | This work |
| | of φC31, Th^{R}, Hyg^{R}, Am^{R} | |
| T149A | T149 with the wilde type accB copy of the | This work |
| | chromosome replaced by the accB::hyg mutant | |
| | allele, Hyg^{R}, Am^{R} | |
| E. coli | | |
| DH5α | F⁻ ΔlacU169 (φ80lacZΔM15) endA1 recA1 | Hanahan (1983) |
| | hsdR17 deoR supE44 thi-I λ⁻gyrA96 relA1 | |
| BL2Iλ(DE3) | F′ ompTr_{B}⁻m_{B}⁻(DE3) | Studier & Moffatt |
| | | (1986) |
| ET 12567 | supE44 hrdS20 (r⁻_{B}m⁻_{B}) ara-14 pro A2 lacY galK2 | MacNeil et al. (1992) |
| | rpsL20 xyl-5 mtl-1 dam⁻ dcm⁻ hsdM Cm^{R} | |
| RG7 | DH5α carrying pCL1 and pBA11 plasmids | Rodriguez & |
| | | Gramajo (1999) |
| Plasmids | | |
| pBluescript SK(+) | Phagemid vector (Ap^{R} lacZ′) | Stratagene |
| pGEM-T Easy | For cloning PCR products | Promega |
| pIJ2925 | pUC18 derivative (Ap^{R} lacZ′) | Janssen & Bibb |
| | | (1993) |
| pSET151 | For the conjugal transfer of DNA from E. coli to | Bierman et al. (1992) |
| | Streptomyces spp. (Ap^{R} Th^{R} lacZ′) | |
| pET22b(+) | Phagemid vector (Ap^{R} lacZ′) for expression of | Novagen |
| | recombinant proteins under control of strong T7 | |
| | transcription and translation signals | |
| pUZ8002 | RK2 derivative with defective oriT (Km^{R}) | Paget et al. (1999) |
| pIJ8600 | For the conjugal transfer of DNA from E. coli to | Sun et al. (1999) |
| | Streptomyces spp. and for expression of | |
| | recombinant proteins under tipA promoter | |
| pBA11 | Vector containing E. coli birA gene | Barker & Campbell |
| | | (1981) |
| pCL1 | pSK(+) with a EcoRI-KpnI insert carrying accA1 | Rodriguez & |
| | | Gramajo (1999) |
| pMR08 | pSK(+) with a SstI insert carrying accBE | This work |
| pTR88 | pET22b(+) with accBE under control of strong T7 | This work |
| | transcription and translation signals | |
| pTR90 | pET22b(+) with accB under control of strong T7 | This work |
| | transcription and translation signals | |
| pTR107 | pET22b(+) with accE under control of strong T7 | This work |
| | transcription and translation signals | |
| pTR124 | pSET151 with a hyg (Hyg^{R}) gene inserted in the | This work |
| | accE coding region | |
| pTR141 | pIJ8600 derivative carrying oriT RK2, ori pUC18, | This work |
| | attP site, int φC31 and aac(3)IV (Am^{R}) | |
| pTR149 | pTR141 with a KpnI insert carrying accBE | This work |

TABLE 2
Heterologous expresion of acyl-CoA carboxylase
components in cell-free extracts of E. coli and in vitro
reconstitution of enzyme activity

| | | Cell-free extracts | |
|---|---|---|---|
| Strain | Proteins | ACCase | PCCase |
| E. coli* | induced by IPTG | [mU (mg protein)⁻¹]⁺ | [mU (mg protein)⁻¹]⁺ |
| RG7 | AccA1 + BirA | ND | ND |
| RG8 | AccB, AccE | ND | ND |
| RG9 | AccB | ND | ND |
| RG10 | AccE | ND | ND |
| RG7:RG8^{&} | AccA1 + BirA:AccB, AccE | 2.35 ± 0.06 | 3.10 ± 0.07 |
| RG7:RG9^{&} | AccA1 + BirA;AccB | 0.32 ± 0.05 | 0.50 ± 0.05 |
| RG7:RG9:RG10^{&} | AccA1 + BirA:AccB;AccE | 1.38 ± 0.05 | 1.77 ± 0.06 |

ND, Not detectable. The amount of ¹⁴C fixed into acid-stable products was not significantly higher than background levels (10-30 c.p.m., equivalent to 0.02-0.06 mU).
*All the RG strains are derived from E. coli DH5α
⁺Results are means of three determinations ± SE
⁺⁺pBA11 expresses BirA constitutively
^{&}Mix of equal amount of proteins from cell-free extracts from each of the strains indicated

TABLE 3
ACCase and PCCase activities in M145, M86 and M94

| Strain | Induction | Activity | |
|---|---|---|---|
| S. | with | ACCase | PCCase |
| coelicolor | Thiostrepton | [mU (mg protein)^{-1]*} | [mU (mg protein)^{-1]*} |
| M145 | − | 1.12 ± 0.03 | 2.2 ± 0.03 |
| M86 | − | 0.43 ± 0.03 | 1.45 ± 0.06 |
| M86 | + | 0.33 ± 0.03 | 0.95 ± 0.06 |
| M94 | − | 0.40 ± 0.03 | 1.57 ± 0.03 |
| M94 | + | 4.61 ± 0.03 (11.5) | 5.41 ± 0.03 (3.5) |

*Results are means of three determinations ± SE.

TABLE 4
Production of actinorhodin and undecylprodigiosin in YEME
medium by M145 and M94.

| | M145 | | M94 | | [M94 + 1 μg Th]* | | [M94 + 5 μg Th]* | |
|---|---|---|---|---|---|---|---|---|
| Time (h) | Act | Red | Act | Red | Act (M) | Red (M) | Act (M) | Red (M) |
| 40 | — | — | — | — | 3 × 10⁻⁷ | 10 × 10⁻⁷ | 11 × 10⁻⁷ | 28 × 10⁻⁷ |
| 60 | — | — | — | — | 9 × 10⁻⁷ | 12 × 10⁻⁷ | 26 × 10⁻⁷ | 78 × 10⁻⁷ |

*Results are mean of two indepent determinations.
(—) Means no detection of the antibiotics.

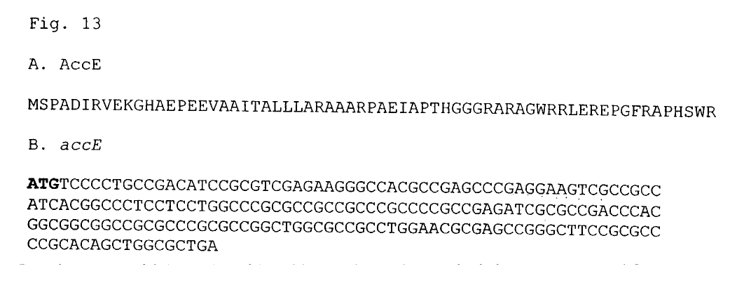

1. A nucleic acid comprising a nucleic acid sequence which encodes AccB polypeptide, or a nucleic acid sequence complementary thereto,
wherein said AccB polypeptide has the amino acid sequence set out in FIG. 12A and/or the amino acid sequence encoded by the nucleic acid sequence set out in FIG. 12B.

2. The nucleic acid of claim 1, wherein said nucleic acid sequence is as set out in FIG. 12B, or is complementary thereto.

3. The nucleic acid of claim 1, further comprising a nucleic acid sequence which encodes AccE polypeptide, or a nucleic acid sequence complementary thereto,
wherein said AccE polypeptide has the amino acid sequence set out in FIG. 13A and/or the amino acid sequence encoded by the nucleic acid sequence set out in FIG. 13B.

4. The nucleic acid of claim 3, wherein said nucleic acid sequence is as set out in FIG. 13B, or is complementary thereto.

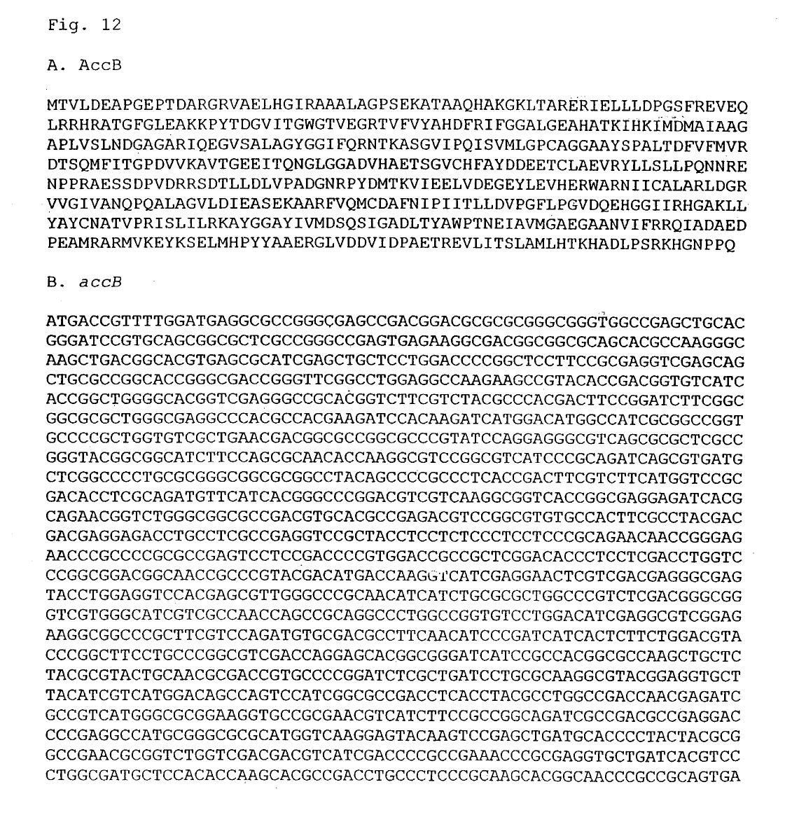

5. The nucleic acid of claim 1, further comprising a nucleic acid sequence which encodes AccA1 polypeptide, or a nucleic acid sequence complementary thereto,
wherein said AccA1 polypeptide has the amino acid sequence set out in FIG. 11A and/or the amino acid sequence encoded by the nucleic acid sequence set out in FIG. 11B.

6. The nucleic acid of claim 5, wherein said nucleic acid sequence is as set out in FIG. 11B, or is complementary thereto.

7. The nucleic acid of claim 1, further comprising a nucleic acid sequence which encodes AccA2 polypeptide, or a nucleic acid sequence complementary thereto,
wherein said AccA2 polypeptide has the amino acid sequence set out in FIG. 11A and/or the amino acid sequence encoded by the nucleic acid sequence set out in FIG. 11B.

8. The nucleic acid of claim 7, wherein said nucleic acid sequence is as set out in FIG. 11B, or is complementary thereto.

9. The nucleic acid of claim 1 wherein said nucleic acid sequence which encodes AccB polypeptide is in operative association with a regulatory sequence for constitutive or inducible expression of said AccB polypeptide in Streptomyces species.

10. The nucleic acid of claim 9 wherein said regulatory sequence comprises the tipA inducible promoter.

11. The nucleic acid of claim 3 wherein said nucleic acid sequence which encodes AccE polypeptide is in operative association with a regulatory sequence for constitutive or inducible expression of said AccE polypeptide in Streptomyces species.

12. The nucleic acid of claim 11 wherein said regulatory sequence comprises the tipA inducible promoter.

13. The nucleic acid of claim 5 wherein said nucleic acid sequence which encodes AccA1 polypeptide is in operative association with a regulatory sequence for constitutive or inducible expression of said AccA1 polypeptide in Streptomyces species.

14. The nucleic acid of claim 13 wherein said regulatory sequence comprises the tipA inducible promoter.

15. The nucleic acid of claim 7 wherein said nucleic acid sequence which encodes AccA2 polypeptide is in operative association with a regulatory sequence for constitutive or inducible expression of said AccA2 polypeptide in Streptomyces species.

16. The nucleic acid of claim 15 wherein said regulatory sequence comprises the tipA inducible promoter.

17. A vector comprising the nucleic acid sequence set out in FIG. 12B under the control of the tipA promoter, whereby said vector is capable, after incorporation into Streptomyces coelicolor, of causing expression of AccB polypeptide having the amino acid sequence set out in FIG. 12A and/or the amino acid sequence encoded by the nucleic acid sequence set out in FIG. 12B.

18. The vector of claim 17, further comprising the nucleic acid sequence set out in FIG. 13B under the control of said tipA promoter, whereby said vector is capable, after incorporation into Streptomyces coelicolor, of causing expression of AccE polypeptide having the amino acid sequence set out in FIG. 13A and/or the amino acid sequence encoded by the nucleic acid sequence set out in FIG. 13B.

19. A Streptomyces coelicolor strain comprising the vector of claim 17.

20. A Streptomyces coelicolor strain comprising the vector of claim 18.

21. A method of producing a polyketide, the method comprising:
providing a polyketide-producing strain of Streptomyces coelicolor into which the vector of claim 17 has been introduced;
culturing said strain under conditions suitable for polyketide synthesis; and
extracting said polyketide from the cell culture medium.

22. The method of claim 21, further comprising the step of purifying said polyketide.

23. The method of claim 22, further comprising the step of formulating said polyketide as a pharmaceutical.

24. The method of claim 21 wherein said polyketide is an antibiotic.

25. A method of producing a polyketide, the method comprising:
providing a polyketide-producing strain of Streptomyces coelicolor into which the vector of claim 18 has been introduced;
culturing said strain under conditions suitable for polyketide synthesis; and
extracting said polyketide from the cell culture medium.

26. The method of claim 25, further comprising the step of purifying said polyketide.

27. The method of claim 26, further comprising the step of formulating said polyketide as a pharmaceutical.

28. The method of claim 27, wherein said polyketide is an antibiotic.

29. A method of modifying a polyketide-producing strain of a Streptomyces species to increase production of said polyketide, the method comprising modifying said strain to express, or to increase expression of, nucleic acid according to claim 1.

30. The method of claim 29, wherein said polyketide is an antibiotic.

31. The method of claim 29, wherein said Streptomyces species is selected from the group consisting of S. coelicolor, S. violaceoruber, S. lividans and S. parvulus.

32. The method of claim 31, wherein said strain is selected from the group consisting of ATCC 12434, ATCC 19832, S. coelicolor A3(2) and S. lividans 66.

33. The method of claim 31, wherein said Streptomyces species is S. coelicolor.

34. The method of claim 32, wherein said strain is S. coelicolor A3 (2).

35. The method of claim 29, further comprising modifying said strain to express, or to increase expression of, nucleic acid according to claim 3.

36. The method of claim 29, further comprising modifying said strain to express, or to increase expression of, nucleic acid according to claim 5.

37. The method of claim 29, further comprising modifying said strain to express, or to increase expression of, nucleic acid according to claim 7.

38. A modified strain of a Streptomyces species, produced according to the method of claim 29.

39. A method of producing a polyketide, the method comprising:
providing the modified Streptomyces strain of claim 38;
culturing said strain under conditions suitable for polyketide synthesis; and
extracting said polyketide from the cell culture medium.

40. The method of claim 39, further comprising the step of purifying said polyketide.

41. The method of claim 40, further comprising the step of formulating said polyketide as a pharmaceutical.

42. A method of increasing acetyl-CoA carboxylase (ACCase) activity in a strain of Streptomyces coelicolor, the method comprising modifying said strain to express, or to increase expression of, nucleic acid encoding AccB polypeptide, said nucleic acid having the nucleic acid sequence set out in FIG. 12B.

43. The method of claim 42, further comprising modifying said strain to express, or to increase expression of, nucleic acid encoding AccE polypeptide, said nucleic acid having the nucleic acid sequence set out in FIG. 13B.

44. The method of claim 42, further comprising modifying said strain to express, or to increase expression of, nucleic acid encoding AccA1 polypeptide, said nucleic acid having the nucleic acid sequence set out in FIG. 11B.

45. The method of claim 42, further comprising modifying said strain to express, or to increase expression of, nucleic acid encoding AccA2 polypeptide, said nucleic acid having the nucleic acid sequence set out in FIG. 11B.

46. The method of claim 42, wherein the strain is S. coelicolor A3(2).

47. A method of increasing production of a polyketide in Streptomyces coelicolor, the method comprising culturing said cells in the presence of exogenous malonate.

48. The method of claim 47, wherein the malonate is present at a concentration of at least about 0.1%.

49. The method of claim 48, wherein the malonate is present at a concentration of at least about 0.5%.

50. The method of claim 49, wherein the malonate is present at a concentration of at least about 1%.

51. The method of claim 47 wherein the polyketide is an antibiotic.
